SEQUENCE LISTING (31 sequences)

SEQ ID NO: 1 - protein, 1125 aa, Bos taurus
MPRAPRCRAVRALLRASYRQVLPLAAFVRRLRPQGHRLVRRGDPAAFRALVAQCLVCVPWDAQPPPAAPSFRQVSCLKELVARVVQRLCERGARNVLAFGFTLLAGARGGPPVAFTTSVRSYLPNTVTDTLRGSGAWGLLLHRVGDDVLTHLLSRCALYLLVPPTCAYQVCGPPLYDLRAAAAAARRPTRQVGGTRAGFGLPRPASSNGGHGEAEGLLEARAQGARRRRSSARGRLPPAKRPRRGLEPGRDLEGQVARSPPRVVTPTRDAAEAKSRKGDVPGPCRLFPGGERGVGSASWRLSPSEGEPGAGACAETKRFLYCSGGGEQLRRSFLLCSLPPSLAGARTLVETIFLDSKPGPPGAPRRPRRLPARYWQMRPLFRKLLGNHARSPYGALLRAHCPLPASAPRAGPDHQKCPGVGGCPSERPAAAPEGEANSGRLVQLLRQHSSPWQVYGLLRACLRRLVPAGLWGSRHNERRFLRNVKKLLSLGKHGRLSQQELTWKMKVQDCAWLRASPGARCVPAAEHRQREAVLGRFLHWLMGAYVVELLRSFFYVTETTFQKNRLFFFRKRIWSQLQRLGVRQHLDRVRLRELSEAEVRQHQEARPALLTSRLRFVPKPGGLRPIVNVGCVEGAPAPPRDKKVQHLSSRVKTLFAVLNYERARRPGLLGASVLGMDDIHRAWRAFVLPLRARGPAPPLYFVKVDVVGAYDALPQDKLAEVIANVLQPQENTYCVRHCAMVRTARGRMRKSFKRHVSTFSDFQPYLRQLVEHLQAMGSLRDAVVIEQSCSLNEPGSSLFNLFLHLVRSHVIRIGGRSYIQCQGIPQGSILSTLLCSFCYGDMENKLFPGVQQDGVLLRLVDDFLLVTPHLTRARDFLRTLVRGVPEYGCQVNLRKTVVNFPVEPGALGGAAPLQLPAHCLFPWCGLLLDTRTLEVHGDHSSYARTSIRASLTFTQGFKPGRNMRRKLLAVLQLKCHGLFLDLQVNSLQTVFTNVYKIFLLQAYRFHACVLQLPFSQPVRSSPAFFLQVIADTASRGYALLKARNAGASLGARGAAGLFPSEAAQWLCLHAFLLKLARHRVTYSRLLGALRTARARLHRQLPGPTRAALEAAADPALTADFKTILD

SEQ ID NO: 2 - protein, 303 aa, Bos taurus
MATSRYEPVAEIGVGAYGTVYKARDPHSGHFVALKSVRVPNGGGAGGGLPISTVREVALLRRLEAFEHPNVVRLMDVCATARTDRETKVTLVFEHVDQDLRTYLDKAPPPGLPVETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFSPRGPRPVQSVVPELEESGAQLLLEMLTFNPHKRISAFRALQHSYLHKAEGDAE

SEQ ID NO: 3 - protein, 708 aa, synthetic construct
MDKVLNREESLQLMDLLGLERSAWGNIPLMRKAYLKKCKEFHPDKGGDEEKMKKMNTLYKKMEDGVKYAHQPDFGGFWDATEIPTYGTDEWEQWWNAFNEENLFCSEEMPSSDDEATADSQHSTPPKKKRKVEDPKDFPSELLSFLSHAVFSNRTLACFAIYTTKEKAALLYKKIMEKYSVTFISRHNSYNHNILFFLTPHRHRVSAINNYAQKLCTFSFLICKGVNKEYLMYSALTRDPFSVIEESLPGGLKEHDFNPEEAEETKQVSWKLVTEYAMETKCDDVLLLLGMYLEFQYSFEMCLKCIKKEQPSHYKYHEKHYANAAIFADSKNQKTICQQAVDTVLAKKRVDSLQLTREQMLTNRFNDLLDRMDIMFGSTGSADIEEWMAGVAWLHCLLPKMDSVVYDFLKCMVYNIPKKRYWLFKGPIDSGKTTLAAALLELCGGKALNVNLPLDRLNFELGVAIDQFLVVFEDVKGTGGESRDLPSGQGINNLDNLRDYLDGSVKVNLEKKHLNKRTQIFPPGIVTMNEYSVPKTLQARFVKQIDFRPKDYLKHCLERSEFLLEKRIIQSGIALLLMLIWYRPVAEFAQSIQSRIVEWKERLDKEFSLSVYQKMKFNVAMGIGVLDWLRNSDDDDEDSQENADKNEDGGEKNMEDSGHETGIDSQSQGSFQAPQSSQSVHDHNQPYHICRGFTCFKKPPTPPPEPET

SEQ ID NO: 4 - protein, 131 aa, unidentified
MLSGGGGDADLANAAARGQVEAVRQLLEAGVDPNRLNRFGRRPIQVMMMGSARVAELLLLHGADPNCADPATLTRPVHDAAREGFLDTLVALHRAGGRLDVRDAWGRLPVDLAEERGHRDVARYLRATAGD

SEQ ID NO: 5 - protein, 150 aa, Bos taurus
MVRRFLITVRIRRANGPPRVRIFVVHIARAAGEWAAPSVRAAVALVLMASEEPAQSAAMHPRPGDDDGQRPRGRAAAAPRRGPQLRGPRHPHPTGARRRPGGLPGHAGGPAPSWSAAGCARCLGPPARGPGGGAGPPRRRPVPARGCRGH

SEQ ID NO: 6 - protein, 198 aa, Bos taurus
MSNVRVSNGSPSLERMDARQAEYPKPSACRNLFGPVNHEELTRDLEKHCRDMEEASQRKWNFDFKNHKPLEGKYEWQEVEKGSLPEFYHRPPRPPKGACKVPAQEGQDASGARPAVPLLGSQANPEDTHLVDQKTDAPDSQTGLAEQCPGIRKRPAADDSSPQNKRANRTEENVSDGSPNAGSVEQTPKKPGLRRRQT

SEQ ID NO: 7 - protein, 168 aa, Bos taurus
MAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLPVVEFLVKHTACKVGHRNHQGDTACDLARLYRRNEVVSLMEGNRAEGAANLQ

SEQ ID NO: 8 - protein, 386 aa, Bos taurus
MEESQAELNVEPPLSQETFSDLWNLLPENNLLSSELSAPVDDLLPYTDVATWLDECPNEAPQMPEPSAPAAPPPATPAPATSWPLSSFVPSQKTYPGNYGFRLGFLQSGTAKSVTCTYSPSLNKLFCQLAKTCPVQLWVDSPPPPGTRVRAMAIYKKLEHMTEVVRRCPHHERSSDYSDGLAPPQHLIRVEGNLRAEYLDDRNTFRHSVVVPYESPEIDSECTTIHYNFMCNSSCMGGMNRRPILTIITLEDSCGNLLGRNSFEVRVCACPGRDRRTEEENLRKKGQSCPEPPPRSTKRALPTNTSSSPQPKKKPLDGEYFTLQIRGFKRYEMFRELNDALELKDALDGREPGESRAHSSHLKSKKRPSPSCHKKPMLKREGPDSD

SEQ ID NO: 9 - protein, 1131 aa, Sus scrofa
MPRAPRCRAVRSLLRDRYRQVLPLATFVRRLGPEGRRLVRRGDPAAYRALVAQCLVCVPWDAQPPPASPSFRQVSCLKELVARVVQRLCERGARNVLAFGFALLDGARGGPPVAFTTSVRSYLPNTVTDTLRGSGAWGLLLRRVGDDVLTHLLARCALYLLVPPSCAYQVCGPPLYDLYTAAEARPMRHKGQTPTGLGLTRPVCNGEAGRPQEQRAQGVRRRRGRAGGHPLPAKRPRHVPEPEQGPEGQASRAHQGRAPGPSDSDPPVMTPTRAAAKAKSREGEAPGTRHLSPQAGGARGTCPPSWWQPHLQGKPSPHVCAAETKRFLYCSGSKEGLRRSFLLCSLPPSLAGAGRLVEVIFLASKPGQPGARRVPARYWRMRPLFRELLKNHARCPYKALLRAHCPLRAAATLSGSGGQVCDHKVGPLAPERLAAAAEGDSASRRLVQLLRQHSSPWQVYRLLRACLHRLVPPGLWGSPHNKRRFLKNVKKLVSLGKHARLSLQELMWKMKVQDCIWLRRSPDARHVQAAEHRLREAILAKFLRWLMGTYVVELLRSFFYVTETTFQKNRLFFFRKRIWSRLQSAGIRQHLDRVRLRELSEAEIRRRREARPAVLTSKLRFVPKPDGLRPIVNMANVVRARTGPGDKKVRRLTGQVKTLFAVLNYERARRPRLLGASVLGVGDIHRAWRAFVLPLRAQDPAPPLYFVKVDVTGAYDALPQDRLLEVVANVIRPHESTYCVRQCAVLRRTARGHVRKSFQTHVSTFADLQPYMRQFVAHLQATGPLRDAVVIEQSCSLNEAGSRLLELFLSLLRNHVIRIGGRSYVQCQGIPQGSILSTLLCSLCYGDMENRLFPGIQRDGVLLRLVDDFLLVTPHLTRAKAFLRTLVRGVPEYGCLANLRKTAVNFPVEDGARGGPAPLQLPAHCLFPWCGLLLDTRTLEVHCDYASYARTSIRASLTFNQGFKPGRNMRRKLLAVLRLKCHGILLDLQVNSLPTVLANVYKIFLLQAYRFHACVLQLPFRQPLARNPSFFLRLVSDTASCCYSLLKARNAGMSLGARGASGPFPSEAAEWLCLHAFLLKLVRHRVTYSCLLGPLRAARERLCQRLPGATLAALEAAADPALTTDFRTILD

SEQ ID NO: 10 - protein, 1105 aa, Thunnus albacares
MSTADTSRVLDILRSLYRHIQTLEEFADSIVFREGQKAILIQQSDTNRFKSFVRGVFVCCDKELQQVPSCNQICTLPELLAFVLNSLKRKRKRNVLAHGYNLLSLAQEERDADHFKFQGDVTQSAAYIHGSDLWKKVTIRLGTDITRYLFESCSVFVTVPPSCVFQVCGIPVYDRVSMTAASSGFYLQSRYRTHNNTQLGRYRGVVTLKRKRNAGNPTISKKRKRRDDRVQRGKRKRETDQKNEEEVMLCSRKRRRVGQHEPTQETQQLCYEPAEEGQPVLVERTESLKPLENSSKGSKHPIETTTVPLEGGPSWRSGIFPPLPPSQCFIRTLGLLYGGRGMRGFLLNRKKKSAEGSRRLQGQDLVRIVFFEGLAYLNGLERKPKKLPQRFFNMVPLFSQLLRQHRRCPYSKILQRMCPLVEERDGGQVEISSLLPQHCGPHRVYLFVRECLSSVIPLELWGSVHNRLNFFARVRGFLRSSKFERLSLAELMWKMKVNDCDWLKISKTGRFPPSELAYRTRVLGQLLAWLLDGYVVGLVRACFYVTESVGQKNALKFYRQEVWAKLLELAFRGHLSKGQMEELTPAQVASLPKTTVISRLRFIPKTDGMRPITRVVGADAKTRLYGGHVRDLLDMLRACVRSTPSLLGSTVWGMTDIHKVLSSLAPTQKDKPQPLYFVKVDVSGAYESLPHEKLIEVIGHALSPVQDELFTIRRYAKIWADSHEGLRKSFVRQADFLEDNMGSTNMKGFVMSLQKRGVHHAILVEQYFCSDLHGREALQFFTQMLTGSVVQFGKKTYRQCRGIPQGSVVSSLLCCLCYGHMENVLFKDITENKGCLMRLVDDFLLITPDLHEAQSFLKILLAGVPQYGLVVNPQKVVINFQASGGMDSCPGIRILPPHCLFPWCGLLLDTHSLDVYKDYSSYAGLSLRYSLTLGSFHSPGQQMRRKLMAILRLKCHALFLDLKTNSLEAVYKNIYKLVLLHAYRFHVCAQSLPFGQTVAKNPVYFLQMIWDMAEYANELIRLSNKGLMLGSKAQTGIVQYEAVELLFCLSFLLVLSQHRSHYKDLLLHLHKRKRSLERCLGDLRLARVRQATNPRTPVDFLAIQT

SEQ ID NO: 11 - protein, 16 aa, synthetic construct
EARPALLTSRLRFIPK

SEQ ID NO: 12 - protein, 306 aa, Thunnus albacares
MAHGSSIQYEPVAEIGGGAYGTVYKARDTESGQFVALKSVRVQTDQNGLPVSTVREVALLRRLEQFDHPNVVRLMDVCATQRSDQETKVTLVFEHVDQDLKTYLERAPAPGLSPDRIKDLMRQLLCGLAFLHSNRVMHRDLKPENILVTSQGQVKLADFGLARIYSCHMALTPVVVTLWYRPPEVLLQSSYATPVDIWSTGCIFAEMFRRKPLFCGDSEVDQLGKIFQVFGLPPEEEWPTDVTLSRKNFPPLFPRPITDFVPEINEQGAQLLLKMLTFDPFKRISALNALEHPYFRDEETLTQTKS

SEQ ID NO: 13 - DNA, 21 bp, synthetic construct
  source 1..21: mol_type = other DNA
  misc_feature 1..19: note = RNA
  misc_feature 20..21: note = DNA
gggatatttaggagtgtgttt

SEQ ID NO: 14 - DNA, 21 bp, synthetic construct
  source 1..21: mol_type = other DNA
  misc_feature 1..19: note = RNA
  misc_feature 20..21: note = DNA
acacactcctaaatatccctg

SEQ ID NO: 15 - DNA, 21 bp, synthetic construct
  source 1..21: mol_type = other DNA
  misc_feature 1..19: note = RNA
  misc_feature 20..21: note = DNA
ggtcagactaagaaatatttt

SEQ ID NO: 16 - DNA, 21 bp, synthetic construct
  source 1..21: mol_type = other DNA
  misc_feature 1..19: note = RNA
  misc_feature 20..21: note = DNA
aatatttcttagtctgacctc

SEQ ID NO: 17 - DNA, 21 bp, synthetic construct
  source 1..21: mol_type = other DNA
  misc_feature 1..19: note = RNA
  misc_feature 20..21: note = DNA
cgcaccgaatagttacggttt

SEQ ID NO: 18 - DNA, 21 bp, synthetic construct
  source 1..21: mol_type = other DNA
  misc_feature 1..19: note = RNA
  misc_RNA 19..21: note = DNA
accgtaactattcggtgcgtt

SEQ ID NO: 19 - RNA, 19 nt, synthetic construct
  source 1..19: mol_type = other RNA
agaccgaactggtttcgct

SEQ ID NO: 20 - RNA, 19 nt, synthetic construct
  source 1..19: mol_type = other RNA
gaagaaaatttccgcaaaa

SEQ ID NO: 21 - protein, 289 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 22 - protein, 289 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 23 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 24 - protein, 158 aa, Human papillomavirus
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 25 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 26 - protein, 105 aa, Human papillomavirus
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 27 - protein, 439 aa, Bos taurus
MPLNVSFANKNYDLDYDSVQPYFYCDEEENFYHQQQQSELQPPAPSEDIWKKFELLPTPPLSPSRRSGLCSPSYVAVASFSPRGDDDGGGGSFSSADQLEMVTELLGGDMVNQSFICDPDDETLIKNIIIQDCMWSGFSAAAKLVSEKLASYQAARKDGGSPSPARGHGGCSTSSLYLQDLSAAASECIDPSVVFPYPLNDSSSPKPCASPDSTAFSPSSDSLLSSAESSPRASPEPLALHEETPPTTSSDSEEEQEDEEEIDVVSVEKRQPPAKRSESGSPSAGSHSKPPHSPLVLKRCHVSTHQHNYAAPPSTRKDYPAAKRAKLDSGRVLKQISNNRKCASPRSSDTEENDKRRTHNVLERQRRNELKRSFFALRDQIPELENNEKAPKVVILKKATAYILSVQAEQQKLKSEIDVLQKRREQLKLKLEQIRNSCA

SEQ ID NO: 28 - protein, 535 aa, Feline leukemia virus
MSGASSGTAIGAKLFGISSVLGEYRVLIGDEGAGPSRSPSEVSFSVWYQSRAARLVTLCLVASFLVPCLTFLIAEAVMGPGSHRRERPSSGVFHLGAPLNVSFANRNYDLDYDSVQPYFYCDEEENFYQQQQQSELQPPAPSEDIWKKFELLPTPPLSPSRRSGLCSPSYVAFASFSPRGDDDGGGGSFSTADQLEMVTELLGGDMVNQSFICDPDDETFIKNIIIQDCMWSGFSAAAKLVSEKLASYQAARKDSGSPSPARGPGGCPTSSLYLQDLTAAASECIDPSVVFPYPLNDSSSPKPCASPDSAAFSPSSDSLLSSAESSPRASPEPLALHEETPPTTSSDSEEEQEEEKEIDVVSVEKRQPPAKRSESGSPSAGGHSKPPHSPLVLKRCHVPTHQHNYAAPPSTRKDYPAAKRAKLDSGRVLKQISNNRKCISPRSSDTEENDKRRTHNVLERQRRNELKRSFFALRDQIPELENNEKAPKVVILKKATAYILSVQAGEQKLISEKDLLRKRREQLKHKLEQLRNSCA

SEQ ID NO: 29 - protein, 326 aa, Bos taurus
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKINKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNTGELESDSGSDKANSPAGGIPSTSSCLPSPSTPVQSPHPQFPHISSTMNGTSSSPSGNHQSSFANRPRKSSVNGSSATSSG

SEQ ID NO: 30 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 31 - protein, 238 aa, Aequorea victoria
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

(19) United States
(12) Patent Application Publication
(10) Pub. No.: US 2025/0320460 A1
(43) Pub. Date: Oct. 16, 2025

(54) INDEFINITE EXTENSION OF CELL PROLIFERATION VIA THE SUPPLEMENTATION OF TRANSIENT, NON-GENOME MODIFYING FACTORS

(71) Applicant: Trustees of Tufts College, Medford, MA (US)

(72) Inventors: David L. Kaplan, Concord, MA (US); Andrew Stout, Cambridge, MA (US); John Yuen, Jr., Somerville, MA (US); Natalie Rubio, Medford, MA (US)

(21) Appl. No.: 18/709,599

(22) PCT Filed: Nov. 15, 2022
(86) PCT No.: PCT/US2022/079907

§ 371 Date: May 13, 2024

Related U.S. Application Data
(60) Provisional application No. 63/278,479, filed on Nov. 11, 2021.

(51) Int. Cl. C12N 5/077; A23L 13/00

(52) U.S. Cl. CPC C12N 5/0659; A23L 13/00

(57) ABSTRACT

The present disclosure relates to cultured tissue, methods for production of the cultured tissue, e.g., cultured meat, that is not genetically modified. Further, the disclosure provides method of temporary immortalization of cells for propagation in vitro.

### CROSS-REFERENCE TO RELATED APPLICATIONS

The present application is the national stage entry of PCT/US2022/079907, filed Nov. 15, 2022, which is based on, and claims priority to U.S. Provisional Patent Application No. 63/279,479 that was filed Nov. 15, 2021. The entire contents of these applications are incorporated by reference herein.

### SEQUENCE LISTING

A Sequence Listing accompanies this application and is submitted as an xml file of the sequence listing named “166118_01401.xml” which is 40,770 bytes in size and was created on May 15, 2024. The sequence listing is electronically submitted via Patent Center and is incorporated herein by reference in its entirety.

### STATEMENT REGARDING FEDERALLY SPONSORED RESEARCH OR DEVELOPMENT

Not applicable.

### BACKGROUND

The present disclosure generally relates to cultured tissue and to methods for producing cultured tissue without genetically modifying the cultured tissue. The cultured tissue may be cultured meat that resembles whole muscle meat and meat products.

Conventional animal agriculture for the production of meat (muscle and fat tissue) is linked to numerous drawbacks such as environmental degradation, zoonic disease emergence, antimicrobial resistance, and animal welfare concerns. As meat production is predicted to increase over the coming decades, the impact of meat production and consumption on human health and the environment is expected to increase as well. To reduce these negative impacts on animals and the environment, there is increasing interest in producing alternatives to conventional animal meat, e.g., cultured meat.

A limitation of cultured meat is scalability. Small-scale production increases the price of cultured meat alternatives, making such products prohibitively expensive for many consumers. Scalable, replicable, and automated processes for cultured meat production are needed before cultured meat can become a viable alternative for consumers.

Primary cells typically senesce—or cease doubling—shortly after extraction from a host animal. This is a key issue within the fields of cellular agriculture and cultured meat, as it hampers the ability to generate large amounts of biomass from a single cell isolation procedure. A large-scale cultured meat process would require repeated satellite cell isolations, which would run against the goal of minimizing animal use and suffering. Thus, methods for non-genetically modified large-scale expansion of cells for producing cultured meat are needed.

### SUMMARY

The present disclosure provides methods of expanding and propagating cells in vitro without genetic modification or external factors. In an aspect of the current disclosure, methods of expanding a population of cells in culture are provided. In some embodiments, the methods comprise: (a) delivering one or more transient immortalizing factors to the population of cells in an amount sufficient to temporarily induce immortalization in the cell; (b) culturing the cells of step (a) with the one or more immortalizing factor for a sufficient time to allow for cell proliferation. In some embodiments, the population of cells produced in step (b) do not have a modified genome. In some embodiments, the one or more transient immortalizing factors comprise telomerase reverse transcriptase (TERT), cyclin-dependent kinase 4 (CDK4), SV40 T antigen, Epstein-Barr virus (EBV), adenovirus E1 protein, human papillomavirus (HPV) E6 protein, HPV E7 protein, c-myc, v-myc, Ras or a small molecule. In some embodiments, the one or more transient immortalizing factors can be an inhibitor of a factor of cellular senescence comprising inhibitors of p15, p16, p27, p18, or p53. In some embodiments, the one or more transient immortalizing factors comprise mRNA, siRNA, small molecule, plasmid, minicircle or a protein. In some embodiments, the one or more transient immortalizing factors further comprises CRISPRa or CRISPRi. In some embodiments, the population of cells comprises primary cells. In some embodiments, the population of cells comprises muscle cells. In some embodiments, the muscle cells comprise muscle satellite cells. In some embodiments, the cells comprise bovine cells. In some embodiments, the methods further comprise (c) culturing the population of cells produced after step (b) in culture conditions without the one or more immortalizing factor for a sufficient time to produce cells without the exogenous immortalizing factor. In some embodiments, the one or more transient immortalizing factors is at least two immortalizing factors. In some embodiments, the one or more transient immortalizing factors is (a) telomerase reverse transcriptase (TERT) and targets cyclin-dependent kinase 4 (CDK4); (b) TERT and BmiI (p16 inhibitor); (c) TERT+cell cycle inhibitor (p15, p16, CDK4, BMi1, etc.); (d) telomerase extending factor and factor that overcomes G1-S phase cell cycle checkpoint (e.g., p15 inhibitor, p16 inhibitor, CDK3, Bmi1, etc) and (e) combinations thereof. In some embodiments, proliferation of the cells is at least about 50 doublings at a consistent doubling rate compared to non-engineered cells. In some embodiments, proliferation of the cells is at least 50 or 75% of the doubling rate of non-engineered cells.

In another aspect of the current disclosure, an in vitro derived cell population is provided. In some embodiments the cell population is produced by the method comprising: (a) delivering one or more transient immortalizing factors to the population of cells in an amount sufficient to temporarily induce immortalization in the cell; (b) culturing the cells of step (a) with the one or more immortalizing factor for a sufficient time to allow for cell proliferation. In some embodiments, the population of cells produced in step (b) do not have a modified genome. In some embodiments, the one or more transient immortalizing factors comprise telomerase reverse transcriptase (TERT), cyclin-dependent kinase 4 (CDK4), SV40 T antigen, Epstein-Barr virus (EBV), adenovirus E1 protein, human papillomavirus (HPV) E6 protein, HPV E7 protein, c-myc, v-myc, Ras or a small molecule. In some embodiments, the one or more transient immortalizing factors can be an inhibitor of a factor of cellular senescence comprising inhibitors of p15, p16, p27, p18, or p53. In some embodiments, the one or more transient immortalizing factors comprise mRNA, siRNA, small molecule, plasmid, minicircle or a protein. In some embodiments, the one or more transient immortalizing factors further comprises CRISPRa or CRISPRi. In some embodiments, the population of cells comprises primary cells. In some embodiments, the population of cells comprises muscle cells. In some embodiments, the muscle cells comprise muscle satellite cells. In some embodiments, the cells comprise bovine cells. In some embodiments, the methods further comprise (c) culturing the population of cells produced after step (b) in culture conditions without the one or more immortalizing factor for a sufficient time to produce cells without the exogenous immortalizing factor. In some embodiments, the one or more transient immortalizing factors is at least two immortalizing factors. In some embodiments, the one or more transient immortalizing factors is (a) telomerase reverse transcriptase (TERT) and targets cyclin-dependent kinase 4 (CDK4); (b) TERT and BmiI (p16 inhibitor); (c) TERT+cell cycle inhibitor (p15, p16, CDK4, BMi1, etc.); (d) telomerase extending factor and factor that overcomes G1-S phase cell cycle checkpoint (e.g., p15 inhibitor, p16 inhibitor, CDK3, Bmi1, etc) and (e) combinations thereof. In some embodiments, proliferation of the cells is at least about 50 doublings at a consistent doubling rate compared to non-engineered cells. In some embodiments, proliferation of the cells is at least 50 or 75% of the doubling rate of non-engineered cells.

In another aspect of the current disclosure, a foodstuff comprising a population of cells produced by the method comprising: (a) delivering one or more transient immortalizing factors to the population of cells in an amount sufficient to temporarily induce immortalization in the cell; (b) culturing the cells of step (a) with the one or more immortalizing factor for a sufficient time to allow for cell proliferation, wherein the cells are not genetically modified. In some embodiments, the population of cells produced in step (b) do not have a modified genome. In some embodiments, the one or more transient immortalizing factors comprise telomerase reverse transcriptase (TERT), cyclin-dependent kinase 4 (CDK4), SV40 T antigen, Epstein-Barr virus (EBV), adenovirus E1 protein, human papillomavirus (HPV) E6 protein, HPV E7 protein, c-myc, v-myc, Ras or a small molecule. In some embodiments, the one or more transient immortalizing factors can be an inhibitor of a factor of cellular senescence comprising inhibitors of p15, p16, p27, p18, or p53. In some embodiments, the one or more transient immortalizing factors comprise mRNA, siRNA, small molecule, plasmid, minicircle or a protein. In some embodiments, the one or more transient immortalizing factors further comprises CRISPRa or CRISPRi. In some embodiments, the population of cells comprises primary cells. In some embodiments, the population of cells comprises muscle cells. In some embodiments, the muscle cells comprise muscle satellite cells. In some embodiments, the cells comprise bovine cells. In some embodiments, the methods further comprise (c) culturing the population of cells produced after step (b) in culture conditions without the one or more immortalizing factor for a sufficient time to produce cells without the exogenous immortalizing factor. In some embodiments, the one or more transient immortalizing factors is at least two immortalizing factors. In some embodiments, the one or more transient immortalizing factors is (a) telomerase reverse transcriptase (TERT) and targets cyclin-dependent kinase 4 (CDK4); (b) TERT and BmiI (p16 inhibitor); (c) TERT+ cell cycle inhibitor (p15, p16, CDK4, BMi1, etc.); (d) telomerase extending factor and factor that overcomes G1-S phase cell cycle checkpoint (e.g., p15 inhibitor, p16 inhibitor, CDK3, Bmi1, etc) and (e) combinations thereof. In some embodiments, proliferation of the cells is at least about 50 doublings at a consistent doubling rate compared to non-engineered cells. In some embodiments, proliferation of the cells is at least 50 or 75% of the doubling rate of non-engineered cells.

In another aspect of the current disclosure, methods for large scale production of in vitro cultured meat product comprising a population of muscle cells are provided. In some embodiments, the methods comprise: (a) delivering one or more transient immortalizing factors to the population of cells in an amount sufficient to temporarily induce immortalization in the cell; (b) culturing the cells of step (a) with the one or more immortalizing factor for a sufficient time to allow for cell proliferation; and (c) culturing the population of cells produced after step (b) in culture conditions without the one or more immortalizing factor for a sufficient time to produce cells without the exogenous immortalizing factor. In some embodiments, the one or more transient immortalizing factors comprise telomerase reverse transcriptase (TERT), cyclin-dependent kinase 4 (CDK4), Bmi1, or SV40 T antigen. In some embodiments, the one or more transient immortalizing factors can be an inhibitor of a factor of cellular senescence comprising inhibitors of p15, p16, p27, p18, or p53. In some embodiments, the one or more transient immortalizing factors comprise mRNA, siRNA, small molecule, plasmid, minicircle or a protein. In some embodiments, the one or more transient immortalizing factors further comprises CRISPRa or CRISPRi. In some embodiments, the population of cells comprises primary cells. In some embodiments, the population of cells comprises muscle cells. In some embodiments, the muscle cells comprise muscle satellite cells. In some embodiments, the cells comprise bovine cells. In some embodiments, the cells comprise seafood cells.

Other embodiments and examples are described herein.

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Cultured meat (also called in vitro, cultivated, lab grown meat) prepared using tissue and bioengineering techniques in vitro is another alternative to traditional animal agriculture. By directly growing meat (muscle and fat tissue) in vitro, energy and nutrients may be more efficiently focused on the outcome. The time frame to generate cultured meat tissues in vitro is also thought to be faster compared to traditional animal agriculture and may only require weeks as opposed to months or years for pork and beef, for example. Moreover, tight control over cell biology during tissue cultivation, as well as the production process, allows for the fine tuning of nutritional parameters by engineering muscle, fat, or other cells to produce vital nutrients that would otherwise not be found (or found only at low concentrations) in conventional meat. Thus, cultured meat production systems may offer healthier, more efficient, and more environmentally friendly alternatives to animal-derived meats.

In this invention, genetic targets pursued during the immortalization of different cells, particularly primary cells, are used. However, instead of modifying the genome of the host organism or cell via the insertion or deletion of specific DNA sequences that would result in “genetically modified cells”, alternate and temporary factors that produce the same effect are utilized. This method of temporarily inducing an immortalized cell state is termed transient immortalization. Transient immortalization permits cells to return to a wild-type state by halting the administration of any immortalizing factors, while potentially avoiding regulatory and public perception complications associated with direct genetic modification.

Methods to transiently induce an immortal-like state in cultured cells include, but are not limited to, mRNA, siRNA, small molecule, plasmid, minicircle and protein delivery, as well as the delivery of epigenetic modifiers such as CRISPRa and CRISPRi. These methods are used to activate or express genes that promote proliferation, including but not limited to telomerase reverse transcriptase (TERT), cyclin-dependent kinase 4 (CDK4) and SV40 T antigen. Conversely, these methods are also used to inhibit factors in the cell that may inhibit proliferation and cause cellular senescence, including but not limited to p15, p16, p27, p18, p53.

To transiently immortalize muscle satellite cells (e.g., bovine, swine, fish, etc.) as an example, TERT mRNA is delivered to prevent replicative senescence (telomere shortening). Further, in combination with the TERT mRNA, an mRNA targeting CDK4 to overcome cellular senescence caused by growth arrest at the G1-S checkpoint in the cell cycle is also delivered. Once an adequate amount of cell proliferation has been achieved, the delivery of these agents is ceased, allowing the muscle satellite cells to return to their original state and be used in their final application as cells that have not undergone direct genome modification. Similar strategies could be employed for other cells, e.g., fat cells, fibroblasts, epithelial cells, or others.

Methods of Expanding a Population of Cells without Genetic Modification

In one aspect of the current disclosure, methods of expanding a population of cells in culture are provided. In some embodiments, the methods comprise (a) delivering one or more transient immortalizing factors to the population of cells in an amount sufficient to temporarily induce immortalization in the cell; (b) culturing the cells of step (a) with the one or more immortalizing factor for a sufficient time to allow for cell proliferation.

In the context of the current disclosure, “population of cells” refers to the cells that are targeted for non-genome editing transformation allowing for controlled, unlimited expansion. In some embodiments, the population of cells comprises precursor cells that are differentiated and expanded into terminally differentiated muscle, fat, or other cells in an arrangement similar to conventionally produced meat. In other embodiments, the population of cells comprises terminally differentiated cells that are contacted with factors that allow the continual replication of the cells without the induction of senescence. As used herein, “senescence” or “cellular senescence” refers to a process in which cells cease dividing and undergo distinctive phenotypic alterations, including profound chromatin and secretome changes, and tumour-suppressor activation. Hayflick and Moorhead first introduced the term senescence to describe the phenomenon of irreversible growth arrest of human diploid cell strains after extensive serial passaging in culture. See, for example, van Deursen, J. M. Nature. 2014 May 22; 509 (7501): 439-446, which is incorporated herein by reference. In the context of the current disclosure, “cell culture” refers to refers to laboratory methods that enable the growth of eukaryotic or prokaryotic cells in physiological conditions in vitro.

In some embodiments, the population of cells is a population propagated from primary cells. The term “primary cells” refers to cells taken directly from living tissue (e.g., muscle or fat tissue of an animal or a biopsy material) and established for growth in vitro. Primary cells usually have undergone very few population doublings in culture and are therefore more representative of the main functional component of the tissue from which they are derived in comparison to continuous (tumor or artificially immortalized) cell lines. The primary cells may be derived from an animal source described herein. The cells may be from animal source including, without limitation, from bovine, avian (e.g., chicken, quail), porcine, seafood, or murine sources. The cells may also be derived from seafood such as fish (e.g., salmon, tuna, etc.), shellfish (e.g., clams, mussels, and oysters); crustaceans (e.g., lobsters, shrimp, prawns, and crayfish), and echinoderms (e.g., sea urchins and sea cucumbers).

In some embodiments of the current disclosure, the population of cells is grown for the purpose of producing comestible or edible products, otherwise known as lab-grown meat. Therefore, the culture conditions for producing such products must be carefully controlled to ensure the safety and wholesomeness of the resulting product. For example, all culture materials, vessels, growth factors, media, etc. must be carefully selected and controlled to prevent the growth of pathogenic organisms or introduce toxins or pollutants into the product.

In some embodiments, the technology is applied to many diverse areas. For example, there is a constant need for fresh human umbilical vein endothelial cells (HUVECs) in vascularization-related medical research, as they typically have a short/finite lifespan and only grow for 16-18 cell doublings during in vitro culture. A current solution to this is the use of immortalized cells/cell lines such as Human umbilical vein endothelial cells (HUVECs) transfected with TERT or mammary gland epithelial cells transfected with the SV40 T antigen. However, in some situations, the constant expression of immortalization genes can interfere with the behavior and phenotypes of the cultured cells, especially since cell differentiation, e.g., from preadipocytes to adipocytes and endothelial cells to blood vessels, often involves a cessation of cell proliferation. Many researchers also opt for normal (non-immortalized) HUVECs because they are more cost-effective. The transient immortalization approach has many advantages. For example, cells such as HUVECs can be proliferated for extended periods of time, but when it comes time to use the cells in experiments (once a sufficient amount of proliferation as been achieved) immortalization can be ceased to make the cells act more similarly to their in vivo/freshly isolated primary cell counterparts. Moreover, immortalized cell lines are also often only available for popular cell types, while adding transient immortalization related factors (e.g., TERT mRNA) could be performed with any cell.

In another example, cell therapies, e.g., chimeric antigen receptor T cells (CAR-T), would benefit from tight control of proliferation of cells. Similarly, cartilage (chondrocytes), some nerve cells, brain microvascular endothelial cells, and other cells are notoriously challenging to propagate in large numbers, so utilizing the methods disclosed herein is beneficial for these cells as well.

In some embodiments, use of the methods to create transiently immortalized fibroblasts, which are useful and highly robust stromal cells for generating tissue with high extracellular matrix protein content and may have applications in cultured meat, is contemplated. The method is applicable to any cell type in which propagation without genetic modification may be desirable.

In some embodiments, the disclosed methods require “delivering” factors to control the replication and prevent senescence of cells in culture. As used herein, delivering or grammatical variations thereof refer to the process of contacting the cultured cells with the delivered agent such that the agent has the intended effect on the target cell. The instant disclosure is drawn to methods of propagating cells, and in some instances, creating an edible cultured meat product, preferably without any genetic modification of the cultured cells (e.g., non-genetically modified organism, non-GMO).

As used herein, genetic modification refers to changes in the nucleotide sequence of the genome of a cell or organism, either in a coding, i.e., a region which is transcribed into mRNA, or a non-coding region of the genome.

The present invention provides in one aspect methods of using in vitro transcribed mRNA for achieving transient immortalization. In some embodiments, the mRNA has a greater than 80% transfection efficiency, in some instances greater than 90+% transfection efficiency. The transient immortalization does not alter the sequence of the targeted cells/organisms. In some embodiments, the method uses epigenetic modifications via CRISPRa and CRISPRi wherein the genetic information of the cell remains unchanged.

In some embodiments, the polynucleotides of the present disclosure may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., 1986 “Basic Methods in Molecular Biology”). Other methods of transformation include for example, lithium acetate transformation and electroporation (see, e.g., Gietz et al., Nucleic Acids Res. 27:69-74 (1992); Ito et al., J. Bacterol. 153:163-168 (1983); and Becker and Guarente, Methods in Enzymology 194:182-187 (1991)).

In some embodiments, the present disclosure teaches methods for getting exogenous protein, RNA, and DNA into a cell. Various methods for achieving this have been described previously including direct transfection of protein/RNA/DNA or DNA transformation followed by intracellular expression of RNA and protein (Dicarlo, J. E. et al. “Genome engineering in Saccharomyces cerevisiae using CRISPR-Cas systems.” Nucleic Acids Res (2013). doi:10.1093/nar/gkt135; Ren, Z. J., Baumann, R. G. & Black, L. W. “Cloning of linear DNAs in vivo by overexpressed T4 DNA ligase: construction of a T4 phage hoc gene display vector.” Gene 195, 303-311 (1997); Lin, S., Staahl, B. T., Alla, R. K. & Doudna, J. A. “Enhanced homology-directed human genome engineering by controlled timing of CRISPR/Cas9 delivery.” Elife. 3, e04766 (2014)).

In some embodiments, the delivered agent is one or more “transient immortalizing factors”. Traditionally, cells are immortalized by in vitro selection, introduction of genetic modifications, or are isolated from tumors and already possess the ability to indefinitely replicate. As used here, an “immortal” cell line is one that can undergo 25 or more doublings, 30 or more doublings, 40 or more doublings, or 50 or more doublings, or more preferably 100 or more doublings, or most preferably infinite doublings without significantly changing the character of the cells. In some embodiments immortal cells are capable of undergoing >50 doublings at a consistent doubling rate, with an average rate of at least 50 or 75% of the doubling rate of non-engineered cells or is faster than non-engineered cells. The selection of transient immortalizing factors may be critical to the success of the method, as particular cell types may require distinct transcriptional programs to sustain the desired level of differentiation while preventing senescence. Exemplary transient immortalization factors include telomerase reverse transcriptase (TERT), cyclin-dependent kinase 4 (CDK4), and simian virus 40 large T antigen (SV40TA).

TERT is a rate-limiting catalytic subunit of telomerase, which maintains the length of telomeric DNA and chromosomal stability. Thus, TERT plays a pivotal role in cellular immortalization, cancer development and progression. Reactivation of telomerase activity allows cells to overcome replicative senescence and to escape apoptosis, both of which are fundamental steps in the initiation of malignant transformation. Bovine TERT has the sequence SEQ ID NO: 1. Porcine TERT has the sequence SEQ ID NO: 9. Yellowfin tuna TERT has the sequence SEQ ID NO: 10. Sequences of TERT for other animal cells are able to be derived from the art. In some embodiments, compositions are delivered to cells to induce the activation of endogenous TERT, for example SEQ ID NO: 11. See, U.S. Patent App. Pub. No. 20190142894, which is incorporated herein by reference. In some embodiments, compounds are delivered to cells to increase the activity of TERT, for example, cycloastragenol which has the formula:

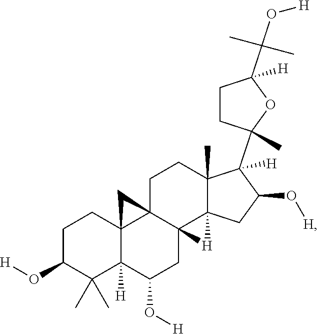

or the pharmaceutical composition known as TA-65.

CDK4, in conjunction with the D-type cyclins, mediates progression through the G1 phase when the cell prepares to initiate DNA synthesis. Bovine CDK4 has the sequence SEQ ID NO: 2. Yellowfin tuna CDK4 has the sequence SEQ ID NO: 12. SV40TA is a key early protein essential for both driving viral replication and inducing cellular transformation in SV40 infection and plays a role in viral genome replication by driving entry of quiescent cells into the cell cycle and by autoregulating the synthesis of viral early mRNA. SV40TA also displays highly oncogenic activities by corrupting the host cellular checkpoint mechanisms that guard cell division and the transcription, replication, and repair of DNA. In addition, SV40TA participates in the modulation of cellular gene expression preceding viral DNA replication. This step involves binding to host key cell cycle regulators retinoblastoma protein RB1/pRb and TP53. SV40TA induces the disassembly of host E2F1 transcription factors from RB1, thus promoting transcriptional activation of E2F1-regulated S-phase genes. SV40 T antigen has the sequence SEQ ID NO: 3.

In some embodiments, viruses, viral proteins, or fragments thereof which are capable of preventing senescence are contemplated as transient immortalization factors. For example, transient immortalization factors comprise Epstein-Barr virus, adenovirus E1 protein (e.g., serotype 2-SEQ ID NO: 21, serotype 5-SEQ ID NO: 22), human papillomavirus (HPV) E6 protein (e.g., type 16-SEQ ID NO: 23, type 18-SEQ ID NO: 23), HPV E7 protein (e.g., type 16-SEQ ID NO: 25, type 18-SEQ ID NO: 26), among others.

In some embodiments, transient immortalization factors comprise c-myc. Bovine c-myc has the sequence SEQ ID NO: 27. Other c-myc sequences are known and understood in the art.

In some embodiments, transient immortalization factors comprise v-myc, which has the sequence SEQ ID NO: 28 when derived from feline leukemia virus, however, other suitable v-myc sequences are contemplated. In some embodiments, transient immortalization factors comprise mutant Ras sequences with that are constitutively active, for example with a glycine to valine substitution at position 12 of Ras (e.g., SEQ ID NO:32 (bovine), Uniprot #P01116 (human), I3LCQ9 (pig), etc). In some embodiments, transient immortalization factors comprise any combination of the foregoing or the factors hereafter. For example, in some embodiments, transient immortalization factors comprise both (1) TERT and (2) c-myc or v-myc.

In some embodiments, transient immortalization factors comprise the protein Polycomb complex protein BMI-1 (BMI-1). BMI-1 antagonizes the function of p16. Bovine BMI-1 has the sequence SEQ ID NO: 29.

In addition, exemplary transient immortalization factors that negatively regulate intrinsic factors within cells are provided including, but not limited to, negative regulators of p15, p16, p27, p18, and p53. Without being bound by any theory or mechanism, such factors are believed to prevent the onset of cellular senescence by interfering with the normal functioning of p15, p16, p27, p18, and p53.

p15, or Cyclin-dependent kinase 4 inhibitor B, also known as multiple tumor suppressor 2 (MTS-2) or p15^{INK4b} is a protein that is encoded by the CDKN2B gene. p15 forms a complex with CDK4 or CDK6, and prevents the activation of the CDK kinases, thus p15 functions as a cell growth regulator that controls cell cycle G1 progression. Bovine p15 has the sequence SEQ ID NO: 4. One skilled in the art is capable of designing and using siRNA to temporarily inhibit p15 expression for the present methods. In some embodiments, siRNA directed to p15 is used in the disclosed methods and compositions as a transient immortalization factor. Exemplary anti-p15 siRNAs are SEQ ID NOs: 13-16. See, for example, Chen, Z. et al. “Targeted inhibition of p57 and p15 blocks transforming growth factor β-inhibited proliferation of primary cultured human limbal epithelial cells”, Mol Vis. 2006 Aug. 23; 12:983-994, which is incorporated by reference herein. p16, also known as p16INK4a, cyclin-dependent kinase inhibitor 2A, CDKN2A, multiple tumor suppressor 1, is a protein that slows cell division by slowing the progression of the cell cycle from the G1 phase to the S phase, thereby acting as a tumor suppressor. It is encoded by the CDKN2A gene. Bovine p16 has the sequence of SEQ ID NO: 5. One skilled in the art is capable of designing and using siRNA to temporarily inhibit p16 expression for the present methods. In some embodiments, inhibitors of p16 are transient immortalization factors, for example the compound SB431542, which has the formula:

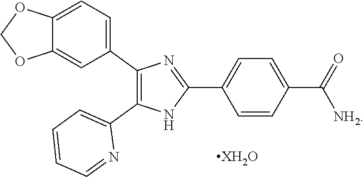 [SMILES: NC(=O)c1ccc(-c2nc(-c3ccc4c(c3)OCO4)c(-c3ccccn3)[nH]2)cc1]

See, for example Mordasky et al. “A small molecule inhibitor of TGFβ1 signaling blocks keratinocyte senescence through inhibition of p16ink4a and p19arf expression”, Cancer Res. May 2008, Volume 68, Issue 9 Supplement. In some embodiments, siRNA targeting p16 is a transient immortalization factor, for example, with sequences SEQ ID NOs: 17 and 18.

p27, also known as p27^{Kip1}, is an enzyme inhibitor that in humans is encoded by the CDKN1B gene. It encodes a protein which belongs to the Cip/Kip family of cyclin dependent kinase (Cdk) inhibitor proteins. The encoded protein binds to and prevents the activation of cyclin E-CDK2 or cyclin D-CDK4 complexes, and thus controls the cell cycle progression at G1. It is often referred to as a cell cycle inhibitor protein because its major function is to stop or slow down the cell division cycle. Bovine p27 has the sequence SEQ ID NO: 6. One skilled in the art is capable of designing and using siRNA to temporarily inhibit p16 expression for the present methods. In some embodiments, transient immortalization factors comprise inhibitors of p27, for example, SJ572403, which has the formula:

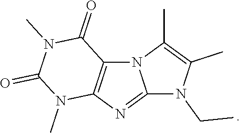 [SMILES: CCn1c(C)c(C)n2c3c(=O)n(C)c(=O)n(C)c3nc12]

In some embodiments, transient immortalization factors comprise siRNAs targeted to p27, for example. See, for example, Akashiba, H. et al. “p27 small interfering RNA induces cell death through elevating cell cycle activity in cultured cortical neurons: a proof-of-concept study”, Cell Mol Life Sci. 2006 October; 63 (19-20): 2397-404, which is incorporated herein by reference. p18, also known as CDKN2C, is a member of the INK4 family of cyclin-dependent kinase inhibitors. This protein has been shown to interact with CDK4 or CDK6, and prevent the activation of the CDK kinases, thus function as a cell growth regulator that controls cell cycle G1 progression. Ectopic expression of this gene was shown to suppress the growth of human cells in a manner that appears to correlate with the presence of a wild-type RB1 function. Bovine p18 has the sequence SEQ ID NO:7. One skilled in the art is capable of designing and using siRNA to temporarily inhibit p18 expression for the present methods. In some embodiments, transient immortalization factors comprise compounds that inhibit p18, for example NSC23005 sodium, which has the formula:

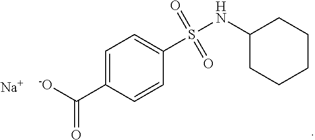

In some embodiments, transient immortalization factors comprise siRNAs targeting p18, for example, SEQ ID NO: 19. See, for example, Matsuzaki et al. “Activation of protein kinase C promotes human cancer cell growth through downregulation of p18INK4c”, Oncogene volume 23, pages 5409-5414 (2004), which is incorporated by reference herein. p53, also known as TP53 or cellular tumor antigen p53, is a tumor suppressor that prevents the outgrowth of aberrant cells, by inducing cell cycle arrest, DNA repair or programmed death. Bovine p53 has the sequence SEQ ID NO: 8. One skilled in the art is capable of designing and using siRNA to temporarily inhibit p53 expression for the present methods. In some embodiments, transient immortalization factors comprise compounds that inhibit p53, for example, pifithrin-«, which has the formula:

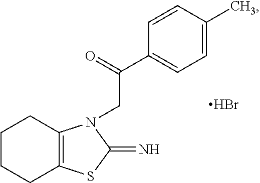 [SMILES: Cc1ccc(C(=O)Cn2c3c(sc2=N)CCCC3)cc1 ; Br]

or similar compounds. In some embodiments, transient immortalization factors comprise siRNAs targeting p53, for example, SEQ ID NO: 20. See, for example, Molitoris et al. “siRNA Targeted to p53 Attenuates Ischemic and Cisplatin-Induced Acute Kidney Injury”, JASN August 2009, 20 (8) 1754-1764, which is incorporated by reference herein. In some aspects, for example, delivery of different combinations of transient immortalizing factors are disclosed herein. In one embodiment, mRNA is delivered to the population of cells. In another embodiment, siRNA is delivered to the population of cells. In another embodiment, nucleic acid sequences (e.g., DNA) encoding the immortalization factors described herein are used. In some embodiments, transient immortalization factors comprise TERT and BMI-1. In some embodiments, transient immortalization factors comprise TERT and an inhibitor of one or more of p15, p16, p27, p18, and p53. In some embodiments, transient immortalization factors comprise one or more of (1) cycloastragenol, TERT, TA-65, TERC and (2) a p15 inhibitor, a p16 inhibitor, CDK4, and BMI-1.

For example, mRNA encoding TERT to prevent telomere shortening and mRNA targeting CDK4 is used to overcome cellular senescence caused by growth arrest at the G1-S checkpoint in the cell cycle. In some embodiments, mRNA may be directly delivered to cells. Suitable modifications must be made to in vitro transcribed (IVT) mRNA such that the mRNA may function correctly when delivered directly to cells. See, for example, Patel S. et al. “Messenger RNA Delivery for Tissue Engineering and Regenerative Medicine Applications” Tissue Eng Part A. 2019 Jan. 1; 25(1-2): 91-112, incorporated herein by reference in its entirety. The use of IVT mRNA is advantageous because mRNA is often reported to have a 90+% transfection efficiency in vitro. Thus, IVT mRNA may encode TERT or SEQ ID NO: 1, CDK4 or SEQ ID NO: 2, SV40TA or SEQ ID NO: 3. In some embodiments, a DNA molecule encoding, for example, TERT or SEQ ID NO: 1, CDK4 or SEQ ID NO: 2, SV40TA or SEQ ID NO: 3 is delivered to the cells. In some embodiments, the DNA molecule comprises a promoter and/or enhancer sequence. In some embodiments, the DNA molecule is a vector.

As used herein, “promoter” refers to a region of DNA where transcription of a gene is initiated. Promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system may also be used. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

In some embodiments, transient immortalization factors comprise muscle-specific promoters which are used to drive expression in muscle cells specifically. See, for example, Wang, B. et al. “Construction and analysis of compact muscle-specific promoters for AAV vectors”, Gene Ther. 15, 1489-1499 (2008); Liu, Y. et al. “Synthetic promoter for efficient and muscle-specific expression of exogenous genes”, Plasmid Vol. 106, November 2019, 102441; and Sarcar et al. “Next-generation muscle-directed gene therapy by in silico vector design”, Nature Comm 10, 492, (2019), which are incorporated herein by reference.

“Enhancer” refers to cis-regulatory elements in the genome that cooperate with promoters to control target gene transcription. Unlike promoters, enhancers are not necessarily adjacent to target genes and can exert their functions regardless of enhancer orientations, positions and spatial segregations from target genes. Therefore, one of skill in the art must select appropriate promoters and, in some embodiments, enhancers to drive expression of proteins encoded on the delivered DNA molecule. See, for example, Meersseman C. et al. “Genetic variability of the activity of bidirectional promoters: a pilot study in bovine muscle”, DNA Research, Volume 24, Issue 3, June 2017, Pages 221-233, incorporated herein by reference, for potential bi-directional promoters active in bovine muscle. See, for example, Kern C. et al. “Functional annotations of three domestic animal genomes provide vital resources for comparative and agricultural research”, Nature Communications volume 12, Article number: 1821 (2021), incorporated herein by reference, for enhancers found in the muscle of chickens, pigs, and cows.

DNA molecules introduced to cells, in methods of the current disclosure, also suitably comprise sequences encoding transient immortalization factors that function by reducing the presence of positive regulators of senescence including, for example, p15, p16, p27, p18, and p53. In some embodiments, the transient immortalization factors are short hairpin RNAs (shRNAs), small interfering RNAs (siRNAs), or other RNA based compositions used in RNA silencing or RNA interference. See, for example, Wilson and Doudna, “Molecular mechanisms of RNA interference”, Annu Rev Biophys. 2013; 42:217-239, incorporated herein by reference in its entirety, for details regarding the mechanisms underlying RNA interference. RNA interference (hereinafter “RNAi”) is a method of post-transcriptional gene regulation that is conserved throughout many eukaryotic organisms. RNAi is induced by short (i.e., <30 nucleotide-long) double stranded RNA (“dsRNA”) molecules which are present in the cell (Fire A et al. (1998), Nature 391:806-811). These short dsRNA molecules, called “short interfering RNA” or “siRNA,” cause the destruction of messenger RNAs (“mRNAs”) which share sequence homology with the siRNA to within one nucleotide resolution (Elbashir S M et al. (2001), Genes Dev, 15:188-200). It is believed that the siRNA and the targeted mRNA bind to an “RNA-induced silencing complex” or “RISC”, which cleaves the targeted mRNA. Thus, in some embodiments, transient immortalization factors comprise combinations of polynucleotides, i.e., mRNA, DNA constructs, e.g., minicircles or other similar minimal plasmid-like DNA molecules, etc. In some embodiments, the transient immortalization factors comprise combinations of protein-encoding polynucleotides and nucleotides encoding siRNAs or shRNAs.

Exogenous expression molecules (polynucleotides) for use the disclosed methods may include one or more externally inducible transcriptional regulatory elements for inducible expression of the one or more transient immortalization factors. For example, polynucleotides useful in the invention may comprise an inducible promoter, such as a promoter that includes a tetracycline response element. In some aspects, the polynucleotide comprises a gene delivery system. Many gene delivery systems are known to those of ordinary skill in the art, and non-limiting examples of useful gene delivery systems include a viral gene delivery system, an episomal gene delivery system, an mRNA delivery system, or a protein delivery system. A viral gene delivery system useful in the invention may be an RNA-based or DNA-based viral vector. An episomal gene delivery system useful in the invention may be a plasmid, an Epstein-Barr virus (EBV)-based episomal vector, a yeast-based vector, a simian virus 40 (SV40)-based episomal vector, a bovine papilloma virus (BPV)-based vector, or the like.

Thus, in some embodiments, the methods of the current disclosure comprise introducing a polynucleotide to a population of cells that comprises a promoter that is inducible by addition of another “induction factor”. In some embodiments, the induction factor is tetracycline. Other suitable induction factors are known in the art including, for example, cumate inducible, rapamycin inducible, FKCsA inducible, Abscisic acid inducible, tamoxifen inducible, blue-light inducible promoters and riboswitches.

In some embodiments, the selected factors disclosed herein are under the control of inducible promoters. In some embodiments, the inducible promoter is a tetracycline inducible promoter (TetON or TetOFF). An exemplary Tet-responsive promoter is described in WO 04/056964A2 (incorporated herein by reference). See, for example, FIG. 1 of WO 04/056964A2. In one construct, a Tet operator sequence (TetOp) is inserted into the promoter region of the vector encoding the disclosed factors. TetOp is preferably inserted upstream of the transcription initiation site, upstream or downstream from the TATA box. In some embodiments, the TetOp is immediately adjacent to the TATA box. The expression of the target protein encoding sequence is, thus, under the control of tetracycline (or its derivative doxycycline, or any other tetracycline analogue). Addition of tetracycline or doxycycline (dox) relieves repression of the promoter by a tetracycline repressor that the host cells are also engineered to express. Thus, in such embodiments, the inducible factor is tetracycline.

In the TetOFF system, a different tet transactivator protein is expressed in the tetOFF host cell. The difference is that Tet/Dox, when bind to an activator protein, is now required for transcriptional activation. Thus, such host cells expressing the activator will only activate the transcription of an shRNA encoding sequence from a TetOFF promoter at the presence of Tet or Dox.

In some embodiments, the selected factors are under the control of a cumate-inducible promoter. See U.S. patent Ser. No. 10/135,362, which is incorporated by reference herein. Thus, in such embodiments, the inducible factor is cumate, or other similar compounds. Other suitable inducible promoter systems are known in the art and can be found in, for example, Kallunki et al. Cells. 2019 August; 8(8): 796, which is incorporated by reference herein. Additional inducible promoter systems include rapamycin, abscisic acid and FK506 binding protein 12-based inducible promoter systems. The invention of the current disclosure provides methods of transiently immortalizing cells without genetic modification. Thus, transient immortalization factors comprise factors capable of CRISPR interference (CRISPRi) and/or CRISPR activation (CRISPRa). The presently disclosed technologies utilize catalytically inactivated (e.g., nickase or dCas) CRISPR endonucleases that have been mutated to no longer generate double DNA stranded breaks, but which are still able to bind to DNA target sites through their corresponding guide RNAs. In some embodiments, the present disclosure refers to these catalytically inactivated CRISPR enzymes as “dead CRISPR”, or “dCRISPR” enzymes. The “dead” modifier may also be used in reference to specific CRISPR enzymes, such as dead Cas9 (dCas9), or dead Cpf1 (dCpf1).

dCRISPR enzymes function by recruiting the catalytically inactivated dCRISPR enzyme to a target DNA sequence via a guide RNA, thereby permitting the dCRISPR enzyme to interact with the host cell's transcriptional machinery for a particular gene.

In some embodiments, The CRISPRi methods of the present disclosure utilize dCRISPR enzymes to occupy target DNA sequences necessary for transcription, thus blocking the transcription of the targeted gene (L. S. Qi et al., “Repurposing CRISPR as an RNA-Guided Platform for Sequence-Specific Control of Gene Expression.” Cell. 152, 1173-1183 (2013); see also L. A. Gilbert et al., “CRISPR-Mediated Modular RNA-Guided Regulation of Transcription in Eukaryotes.” Cell. 154, 442-451 (2013)). In other embodiments, the CRISPRi methods of the present disclosure utilize dCRISPR enzymes translationally fused, or otherwise tethered to one or more transcriptional repression domains, or alternatively utilize modified guide RNAs capable of recruiting transcriptional repression domains to the target site (e.g., tethered via aptamers, as discussed below). In some embodiments, transient immortalization factors comprise CRISPRi that target, for example, p15, p16, p27, p53, or any combination thereof.

In some embodiments, the CRISPRa methods of the present disclosure employ dCRISPR enzymes translationally fused or otherwise tethered to different transcriptional activation domains, which can be directed to promoter regions by guide RNAs. (See A. W. Cheng et al., “Multiplexed activation of endogenous genes by CRISPR-on, an RNA-guided transcriptional activator system.” Cell Res. 23, 1163-1171 (2013); see also L. A. Gilbert et al., “Genome-Scale CRISPR-Mediated Control of Gene Repression and Activation.” Cell. 159, 647-661 (2014)). In other embodiments, the CRISPRa methods of the present disclosure utilize modified guide RNAs that recruit additional transcriptional activation domains to upregulate expression of the target gene (e.g., tethered via aptamers, as discussed below). In some embodiments, transient immortalization factors comprise CRISPRa that target TERT, CDK4, Bmi1 (Bmi1 is a protein that inhibits p16), or any combination thereof.

In yet other embodiments, the presently disclosed invention also envisions exploiting dCRISPR enzymes and guide RNAs to recruit other regulatory factors to target DNA sites. In addition to recruiting transcriptional repressor or activation domains, as discussed above, the dCRISPR enzymes and guide RNAs of the present disclosure can be modified so as to recruit proteins with activities ranging from DNA methylation, chromatin remodelers, ubiquitination, sumoylation. Thus, in some embodiments, the dCRISPR enzymes and guide RNAs of the present disclosure can be modified to recruit factors with methyltransferase activity, demethylase activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, sumoylating activity, desumoylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, demyristoylation activity, cytidine deaminase activity and any combinations thereof.

In other embodiments, the dCRISPR enzymes and guide RNAs of the present disclosure can be modified to recruit one or more marker genes/composition, such as fluorescent proteins, gold particles, radioactive isotopes, GUS enzymes, or other known biological or synthetic compositions capable of being detected. This last embodiment would permit researchers to tag and track regions of a host cell's genome. As used herein, the term “cis regulatory factors” refers to any of the biological or synthetic compositions that can be recruited by the dCRISPR or guide RNAs of the present disclosure.

In some embodiments, the dCRISPR enzyme and the transcriptional modulator domain are linked via a peptide linker. A peptide linker sequence may be employed to separate the first and the second peptide components by a distance sufficient to ensure that each peptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional regions on the first and second peptides; and (3) the lack of hydrophobic or charged residues that might react with the peptide functional regions. In certain embodiments, the peptide linker sequences contain Gly, Asn and Ser residues. Other near neutral amino acids, such as Thr and Ala may also be used in the linker sequence.

In some embodiments, the present disclosure teaches the use of protein-protein interaction domains to tether the transcriptional modulator domains to the dCRISPR. Thus, the sequence of the dCRISPR enzyme is, for example, translationally fused to a first protein-protein interaction domain (PP1) capable of dimerizing with a second protein-protein interaction domain (PP2) that is translationally fused to the transcriptional modulator (or other cis regulatory factor). When expressed, each of the dCRISPR-PP1 and the PP2-Transcriptional Modulator will dimerize, thus recruiting the transcriptional modulator to the DNA target site. Persons having skill in the art will be aware of methods of using naturally occurring, or synthetic protein-protein interaction domains to create in-vivo dimers. (See Giescke et al., 2006 “Synthetic protein-protein interaction domains created by shuffling Cys2His2 zinc-fingers.” Mol Syst Biol 2:2006.0011).

In other embodiments, the present disclosure also teaches modified guide RNAs with RNA aptamers capable of recruiting one or more cis regulatory factors. The RNA aptamers of the present disclosure may be operably linked to the 5′ or 3′ end of a guide RNA and are designed to not affect dCRISPR binding to a DNA target site. Instead, the RNA aptamers provide an additional tether from which to recruit one or more cis regulatory factors, such as transcriptional modulators.

In some embodiments, the present disclosure teaches customized RNA aptamers designed to directly interact with one or more cis regulatory factors. In other embodiments, the present disclosure teaches use of known aptamers targeting specific sequences. Thus, in some embodiments, the present disclosure envisions guide RNAs with validated RNA aptamers, which then bind to their natural targets, which are in turn translationally fused to one or more cis regulatory factor (i.e., guide_RNA-Aptamer-Aptamer_Target-Cis_Regulatory_Factor). In some embodiments, guide RNAs that incorporate RNA aptamers to tether cis regulatory factors are referred to as scaffold RNAs (scRNAs). (Zalatan J G, et al. “Engineering complex synthetic transcriptional programs with CRISPR RNA scaffolds.” Cell. 2015; 160:339-350). The scRNAs are designed by extending the guide RNA sequence with orthogonally acting protein-binding RNA aptamers. Each scRNA can encode information both for DNA target recognition and for recruiting a specific repressor or activator protein. By changing the DNA targeting sequence or the RNA aptamers in a modular fashion, multiple dCas9-scRNAs can simultaneously activate or repress multiple genes in the same cell

For example, an improvement, termed the synergistic activation mediator (SAM) system, was achieved by adding MS2 aptamers to a guide RNA. The MS2 aptamers were designed to recruit cognate MS2 coat protein (MCP), which were fused to p65AD and heat shock factor 1 (HSF1) (Dominguez et al., 2016 “Beyond editing; repurposing CRISPR-Cas9 for precision genome regulation and interrogation” Nat Rev Mol Cel Biol January 17(1) 5-15). The SAM technology, together with dCas9-VP64, further increased endogenous gene activation compared with dCas9-VP64 alone and was shown to activate 10 genes simultaneously. (Konermann S, et al. “Genome-scale transcriptional activation by an engineered CRISPR-Cas9 complex.” Nature. 2014; 517:583-588). Similar results may be achieved using other validated aptamer-scaffold protein combinations, such as PP7 or com. (Zalatan J G, et al. “Engineering complex synthetic transcriptional programs with CRISPR RNA scaffolds.” Cell. 2015; 160:339-350).

In some embodiments, the present disclosure also envisions the use of double-sided aptamers capable of tethering a dCRISPR enzyme to one or more cis regulatory factors. The double-sided aptamers of the present disclosure function similarly to the aptamers discussed above, but are capable of binding both the dCRISPR protein, and the cis regulatory factor. In one illustrative example, the dCRISPR enzyme would be translationally fused to an MS2 coat protein domain, and the cis regulatory element (a VP16 domain) would be translationally fused to a PP7 domain. The double-sided RNA aptamer would comprise an MS2 binding domain on one end, and a PP7 binding domain on another end. Thus, in some embodiments, the double-sided aptamers of the present disclosure can would be expected to form the following generic structure: dCRISPR-Aptamer_Target-Aptamer_Side1-Aptamer_Side2-Aptamer_Target-Cis_Regulatory Factor.

A non-limiting list of the transcriptional activation domains compatible with the presently disclosed invention include: fragments of transcription regulatory domains and fragments of domains having transcription regulation function of VP16, VP64, VP160, EBNA2, E1A, Gal4, Oaf1, Leu3, Rtg3, Pho4, Gln3, Gcn4, Gli3, Pip2, Pdr1, Pdr3, Lac9, Teal, p53, NFAT, Sp1 (e.g., Sp1a), AP-2 (e.g., Ap-2a), Sox2, NF-κB, MLL/ALL, E2A, CREB, ATF, FOS/JUN, HSF1, KLF2, NF-1L6, ESX, Oct1, Oct2, SMAD, CTF, HOX, Sox2, Sox4, VPR, RpoZ, or Nanog. In some embodiments the transcriptional activator is VPR (see Kiani S. et al., “Cas9 gRNA engineering for genome editing, activation and repression” Nature Methods 12, 1051-1054 (2015)).

A non-limiting list of the transcriptional repressors compatible with the presently disclosed invention include: Mxi1, Tbx3, KRAB (Kruppel-associated box, Margolin, J. F, et al. “Kruppel-associated boxes are potent transcriptional repression domains.” Proc. Natl. Acad. Sci. USA 91, 4509-4513 (1994)), EnR, or SID, SID4X (a tandem repeat of four SID domains linked by short peptide linkers), PIE-1, IAA28-RD among others.

In some embodiments, the nucleic acids encoding for the dCRISPR enzyme and/or the guide RNA are contained in one or more insert parts of a modular CRISPR construct of the present disclosure. Thus, the modular CRISPR constructs of the present disclosure permit users to quickly and efficiently modify the construct to add or subtract insert parts encoding for different guide RNAs (e.g., guide RNAs targeting different genes, or encoding aptamers capable of recruiting different cis regulatory factors, as discussed above), or encoding different dCRISPR enzymes (e.g., dCas9, or dCpf1, or dCRISPR protein fusions with various cis regulatory factors, as discussed above).

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities and encompasses partially purified or substantially purified. Purity may be denoted by a weight-by-weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

In some embodiments, the present disclosure teaches the use of origins of replication to maintain (i.e., continue to replicate) a plasmid in one or more species. Persons having skill in the art will be familiar with various available origin of replication sequences. Common features of origins of replications for bacterial, archael, eukaryotic, and multicellular organisms is discussed in Leonard and Mechali, “DNA replication Origins” Cold Spring Harb Perspect Biol 2013 October; 5 (10).

In some embodiments, the polynucleotides of the current disclosure comprise “selection markers”. Selection markers are factors encoded by the polynucleotide that allow selection of a cell harboring the polynucleotide. In some embodiments, selection markers are, for example, fluorescent proteins or luminescent proteins. In some embodiments, selection markers are polynucleotide sequences that encode proteins that confer resistance to antibiotics. In some embodiments, polynucleotides comprise multiple selection markers. Exemplary selection markers include puromycin, blasticidin, zeocin, G418, or hygromycin resistance. Puromycin resistance is conferred by expression of the protein with amino acid sequence SEQ ID NO: 30. Green fluorescent protein (GFP) has the sequence SEQ ID NO: 31. In addition, auxotrophy may be used as a selection marker. By way of example, but not by way of limitation, auxotrophic cells that are unable to synthesize tyrosine de novo by be delivered a transient immortalization factor that comprises enzyme(s) responsible for such de novo synthesis, thereby allowing selection of cells that comprise the transient immortalization factors using media lacking tyrosine.

### Methods for Large Scale Production of In Vitro Cultured Meat Product

In another aspect of the current disclosure, methods for large scale production of in vitro cultured meat product comprising a population of muscle cells are provided. In some embodiments, the methods comprise: (a) delivering one or more transient immortalizing factors to the population of cells in an amount sufficient to temporarily induce immortalization in the cell; (b) culturing the cells of step (a) with the one or more immortalizing factor for a sufficient time to allow for cell proliferation; and (c) culturing the population of cells produced after step (b) in culture conditions without the one or more immortalizing factor for a sufficient time to produce cells without the exogenous immortalizing factor.

In some embodiments, the meat produced by the methods of the invention may be intended for consumption by human beings, non-human animals, or both. In some embodiments, the cultured meat products are food products for human consumption. In other embodiments, the cultured meat products are used for animal feed such as feed for livestock, feed for aquaculture, or feed for domestic pets.

In some embodiments, the method includes culturing myoblasts in vitro or ex vivo and allowing these cells to differentiate into specific types of muscle cells such as skeletal muscle cells or smooth muscle cells.

In some embodiments, the meat product comprises muscle cells including skeletal muscle cells, smooth muscle cells and satellite cells. In some embodiments, the meat product comprises fat cells (e.g., adipocytes). In some embodiments, the meat product comprises an extra cellular matrix secreted by specialized cells (e.g., fibroblasts). In some embodiments, the meat product comprises endothelial cells or capillary endothelium formed by endothelial cells, including, but not limited to aortic endothelial cells and skeletal microvascular endothelial cells. The meat product may further comprise an extracellular matrix. The meat product may further comprise adipocytes or further comprise capillaries.

The cells may be edible cells including muscle cells, fat cells, and combinations thereof. The precursor cells may be muscle precursor cells or adipocyte precursor cells. Examples of suitable cell types include, but are not limited to, satellite cells, fat cells (i.e., adipocytes), fibroblasts, myoblasts, muscle cells, precursors thereof, and combinations thereof. The cells may be derived from primary cells of suitable animals, as described herein.

The cells may be from animal source including, without limitation, from bovine, avian (e.g., chicken, quail), porcine, seafood, or murine sources. The cells may also be derived from seafood such as fish (e.g., salmon, tuna, etc.), shellfish (e.g., clams, mussels, and oysters); crustaceans (e.g., lobsters, shrimp, prawns, and crayfish), and echinoderms (e.g., sea urchins and sea cucumbers). The cells may be engineered to produce vital nutrients such as proteins and essential fatty acids.

Media formulations may include transgenic components to drive cell differentiation. For example, tetracycline-responsive promoters inserted into transgenic cells may be activated by including tetracycline in the culture medium, resulting in forced expression of myogenic or adipogenic genes in edible cell lines (e.g., chicken fibroblasts, bovine satellite cells, etc.).

Bovine satellite cells may be cultured in growth media with growth factors (e.g., DMEM with Glutamax, 20% FBS, and 1% antiobiotic-antimycotic, and 1 ng/mL human fibroblast growth factor 2 (FGF-2)). To differentiate satellite cells into mature myotubes, cells may be cultured to confluence and triggered for differentiation by a low growth factor environment. For example, the culture medium may shift from a growth factor-rich proliferation media to a growth factor-poor differentiation media.

Bovine fat cells may also be cultured in growth media (e.g., DMEM with Glutamax, 20% FBS, 1% antibiotic-antimycotic). To differentiate adipogenic precursor cells into mature adipocytes, cells may be cultured to a desired confluence (e.g., 75%), and the media may then be supplemented with free fatty acid solution. An exemplary free fatty acid solution may be 50 millimolar (mM) free fatty acid solutions containing elaidic acid, erucic acid, myristoleic acid, oleic acid, palmitoleic acid, phytanic acid, and pristanic acid. To verify lipid accumulation, Oil Red O (ORO) may be used to stain differentiated cells.

Growth factors that can be used in the methods and compositions of the invention include but are not limited to platelet-derived growth factors (PDGF), insulin-like growth factor (IGF-1). PDGF and IGF-1 are known to stimulate mitogenic, chemotactic and proliferate (differentiate) cellular responses. The growth factor can be, but is not limited to, one or more of the following: PDGF, e.g., PDGF AA, PDGF BB; IGF, e.g., IGF-I, IGF-II; fibroblast growth factors (FGF), e.g., acidic FGF, basic FGF, B-endothelial cell growth factor, FGF 4, FGF 5, FGF 6, FGF 7, FGF 8, and FGF 9; transforming growth factors (TGF), e.g., TGF-P1, TGF β1.2, TGF-β2, TGF-β3, TGF-β5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor.

The methods of the current disclosure may comprise culturing the cells in conditions without the one or more immortalizing factor for a sufficient time to produce cells without the exogenous immortalizing factor. Thus, after the meat product has been grown for a sufficient period to produce the desired quantity of meat product, the cells are washed of the transient immortalization factors by culturing the cells for a suitable time in medium without the transient immortalization factors, e.g., 1, 2, 3, 4, 5, 6, 7, 8, 9, 10, 11, 12, 13, or 14 days or more. The transient immortalization factors may comprise selection markers. Thus, one of skill in the art may monitor the meat product for presence of the transient immortalization factors by detecting the presence of the selection marker. A suitable time for the culturing of the cells without the transient immortalization factors is such that the selection marker which comprises the transient immortalization factor is no longer detectable in the cells or meat product by assays known in the art, for example, using an appropriate enzyme-linked immunosorbent assay (ELISA) or PCR/RT-PCR to detect the transient immortalization factors, whichever is appropriate. For example, if the transient immortalization factor is a protein, an ELISA assay may be appropriate to detect the factor, whereas if the factor is an mRNA, RT-PCR may be an appropriate assay to detect the factor. Design or purchase of appropriate assays is well within the ordinary skill in the art.

Various cost-effective biopolymers or complex extracts from natural sources may be used as coating materials. In some embodiments, extracellular matrix proteins and/or chemical/synthetic coatings may be used as coatings to improve cell attachment to the culture vessel and mimic in vivo cell behavior. Other types of coating materials may include commercially available products such as, but not limited to, fibronectin, laminin, vitronectin, collagen, cadherin, elastin, hyaluronic acid, poly-D-lysine, poly-L-lysine, poly-L-ornithine, concanavalin A, and other adhesive, non-toxic chemicals. Conconavalin A, laminin, and hyaluronic acid may be obtained from animal-free origins and have been shown to enhance muscle cell attachment to various biomaterials.

The structural hierarchy and marbling of the cultured tissue construct may be tunable by changing the ratio of muscle cell fibers and fat cell fibers. Warner-Bratzler shear force test may be used to assess the texture and tenderness of the cultured tissue product.

According to the present disclosure, cultured muscle provides versatile outputs that meet target metrics pertaining to properties such as texture, thermal response upon cooking, composition, nutrition, density, alignment, composition, and marbling. This cultured meat system is cost-efficient, scalable, and generates cultured meats that mimic whole muscle.

PCT application number US2021/071171 describes methods and systems to generate whole muscle meat in culture. Accordingly, PCT application number US2021/071171, filed Aug. 12, 2021, is incorporated herein by reference in its entirety. The methods of transiently expanding a population of cells in culture may be used in the bioreactors described in PCT application number US2021/071171. Thus, the instantly disclosed cells and methods for developing cultured cell populations without genetically modifying the cells are suitable for use in the systems and methods disclosed in PCT application number US2021/071171.

As used herein the terms “ingestible” and “edible” refer to compositions which can be safely taken into the body orally. These compositions include those which are absorbed, and those which are not absorbed as well as those which are digestible and non-digestible. As used herein, the term “chewable” refers to a composition which can be broken/crushed into smaller pieces by chewing prior to swallowing. One skilled in the art will appreciate that a suitable edible composition may be selected according to physical properties (e.g., Young's modulus, viscosity modulus, stiffness, etc.) to a desired use (e.g., consumption by a human adult).

TABLE 1
Informal Sequence listing:

| SEQ | | | |
|---|---|---|---|
| ID | | | |
| NO: | Name | Organism | Sequence |
| 1 | Telomerase | Bos | MPRAPRCRAV RALLRASYRQ VLPLAAFVRR LRPQGHRLVR RGDPAAFRAL VAQCLVCVPW 60 |
| | reverse | taurus | DAQPPPAAPS FRQVSCLKEL VARVVQRLCE RGARNVLAFG FTLLAGARGG PPVAFTTSVR 120 |
| | transcrip- | | SYLPNTVTDT LRGSGAWGLL LHRVGDDVLT HLLSRCALYL LVPPTCAYQV CGPPLYDLRA 180 |
| | tase | | AAAAARRPTR QVGGTRAGFG LPRPASSNGG HGEAEGLLEA RAQGARRRRS SARGRLPPAK 240 |
| | | | RPRRGLEPGR DLEGQVARSP PRVVTPTRDA AEAKSRKGDV PGPCRLFPGG ERGVGSASWR 300 |
| | | | LSPSEGEPGA GACAETKRFL YCSGGGEQLR RSFLLCSLPP SLAGARTLVE TIFLDSKPGP 360 |
| | | | PGAPRRPRRL PARYWQMRPL FRKLLGNHAR SPYGALLRAH CPLPASAPRA GPDHQKCPGV 420 |
| | | | GGCPSERPAA APEGEANSGR LVQLLRQHSS PWQVYGLLRA CLRRLVPAGL WGSRHNERRF 480 |
| | | | LRNVKKLLSL GKHGRLSQQE LTWKMKVQDC AWLRASPGAR CVPAAEHRQR EAVLGRELHW 540 |
| | | | LMGAYVVELL RSFFYVTETT FQKNRLFFFR KRIWSQLQRL GVRQHLDRVR LRELSEAEVR 600 |
| | | | QHQEARPALL TSRLRFVPKP GGLRPIVNVG CVEGAPAPPR DKKVQHLSSR VKTLFAVLNY 660 |
| | | | ERARRPGLLG ASVLGMDDIH RAWRAFVLPL RARGPAPPLY FVKVDVVGAY DALPQDKLAE 720 |
| | | | VIANVLQPQE NTYCVRHCAM VRTARGRMRK SFKRHVSTFS DFQPYLRQLV EHLQAMGSLR 780 |
| | | | DAVVIEQSCS LNEPGSSLEN LFLHLVRSHV IRIGGRSYIQ CQGIPQGSIL STLLCSFCYG 840 |
| | | | DMENKLFPGV QQDGVLLRLV DDFLLVTPHL TRARDFLRTL VRGVPEYGCQ VNLRKTVVNF 900 |
| | | | PVEPGALGGA APLQLPAHCL FPWCGLLLDT RTLEVHGDHS SYARTSIRAS LTFTQGFKPG 960 |
| | | | RNMRRKLLAV LQLKCHGLFL DLQVNSLQTV FTNVYKIFLL QAYRFHACVL QLPFSQPVRS 1020 |
| | | | SPAFFLQVIA DTASRGYALL KARNAGASLG ARGAAGLFPS EAAQWLCLHA FLLKLARHRV 1080 |
| | | | TYSRLLGALR TARARLHRQL PGPTRAALEA AADPALTADF KTILD 1125 |
| 2 | Cyclin | Bos | MATSRYEPVA EIGVGAYGTV YKARDPHSGH FVALKSVRVP NGGGAGGGLP ISTVREVALL 60 |
| | dependent | taurus | RRLEAFEHPN VVRLMDVCAT ARTDRETKVT LVFEHVDQDL RTYLDKAPPP GLPVETIKDL 120 |
| | kinase 4 | | MRQFLRGLDF LHANCIVHRD LKPENILVTS GGTVKLADFG LARIYSYQMA LTPVVVTLWY 180 |
| | | | RAPEVLLQST YATPVDMWSV GCIFAEMFRR KPLFCGNSEA DQLGKIFDLI GLPPEDDWPR 240 |
| | | | DVSLPRGAFS PRGPRPVQSV VPELEESGAQ LLLEMLTFNP HKRISAFRAL QHSYLHKAEG 300 |
| | | | DAE 303 |
| 3 | SV40 T | Syn- | MDKVLNREES LQLMDLLGLE RSAWGNIPLM RKAYLKKCKE FHPDKGGDEE KMKKMNTLYK 60 |
| | antigen | thetic | KMEDGVKYAH QPDFGGFWDA TEIPTYGTDE WEQWWNAFNE ENLFCSEEMP SSDDEATADS 120 |
| | | | QHSTPPKKKR KVEDPKDFPS ELLSFLSHAV FSNRTLACFA IYTTKEKAAL LYKKIMEKYS 180 |
| | | | VTFISRHNSY NHNILFFLTP HRHRVSAINN YAQKLCTFSF LICKGVNKEY LMYSALTRDP 240 |
| | | | FSVIEESLPG GLKEHDENPE EAEETKQVSW KLVTEYAMET KCDDVLLLLG MYLEFQYSFE 300 |
| | | | MCLKCIKKEQ PSHYKYHEKH YANAAIFADS KNQKTICQQA VDTVLAKKRV DSLQLTREQM 360 |
| | | | LTNRFNDLLD RMDIMFGSTG SADIEEWMAG VAWLHCLLPK MDSVVYDFLK CMVYNIPKKR 420 |
| | | | YWLFKGPIDS GKTTLAAALL ELCGGKALNV NLPLDRLNFE LGVAIDQFLV VFEDVKGTGG 480 |
| | | | ESRDLPSGQG INNLDNLRDY LDGSVKVNLE KKHLNKRTQI FPPGIVTMNE YSVPKTLQAR 540 |
| | | | FVKQIDFRPK DYLKHCLERS EFLLEKRIIQ SGIALLLMLI WYRPVAEFAQ SIQSRIVEWK 600 |
| | | | ERLDKEFSLS VYQKMKFNVA MGIGVLDWLR NSDDDDEDSQ ENADKNEDGG EKNMEDSGHE 660 |
| | | | TGIDSQSQGS FQAPQSSQSV HDHNQPYHIC RGFTCFKKPP TPPPEPET 708 |
| 4 | p15 | Bos | MLSGGGGDAD LANAAARGQV EAVRQLLEAG VDPNRLNRFG RRPIQVMMMG SARVAELLLL 60 |
| | | taurus | HGADPNCADP ATLTRPVHDA AREGFLDTLV ALHRAGGRLD VRDAWGRLPV DLAEERGHRD 120 |
| | | | VARYLRATAG D 131 |
| 5 | p16 | Bos | MVRRFLITVR IRRANGPPRV RIFVVHIARA AGEWAAPSVR AAVALVLMAS EEPAQSAAMH 60 |
| | | taurus | PRPGDDDGQR PRGRAAAAPR RGPQLRGPRH PHPTGARRRP GGLPGHAGGP APSWSAAGCA 120 |
| | | | RCLGPPARGP GGGAGPPRRR PVPARGCRGH 150 |
| 6 | p27 | Bos | MSNVRVSNGS PSLERMDARQ AEYPKPSACR NLFGPVNHEE LTRDLEKHCR DMEEASQRKW 60 |
| | | taurus | NFDFKNHKPL EGKYEWQEVE KGSLPEFYHR PPRPPKGACK VPAQEGQDAS GARPAVPLLG 120 |
| | | | SQANPEDTHL VDQKTDAPDS QTGLAEQCPG IRKRPAADDS SPQNKRANRT EENVSDGSPN 180 |
| | | | AGSVEQTPKK PGLRRRQT 198 |
| 7 | p18 | Bos | MAEPWGNELA SAAARGDLEQ LTSLLQNNVN VNAQNGFGRT ALQVMKLGNP EIARRLLLRG 60 |
| | | taurus | ANPDLKDRTG FAVIHDAARA GFLDTLQTLL EFQADVNIED NEGNLPLHLA AKEGHLPVVE 120 |
| | | | FLVKHTACKV GHRNHQGDTA CDLARLYRRN EVVSLMEGNR AEGAANLQ 168 |
| 8 | p53 | Bos | MEESQAELNV EPPLSQETFS DLWNLLPENN LLSSELSAPV DDLLPYTDVA TWLDECPNEA 60 |
| | | taurus | PQMPEPSAPA APPPATPAPA TSWPLSSFVP SQKTYPGNYG FRLGFLQSGT AKSVTCTYSP 120 |
| | | | SLNKLFCQLA KTCPVQLWVD SPPPPGTRVR AMAIYKKLEH MTEVVRRCPH HERSSDYSDG 180 |
| | | | LAPPQHLIRV EGNLRAEYLD DRNTFRHSVV VPYESPEIDS ECTTIHYNFM CNSSCMGGMN 240 |
| | | | RRPILTIITL EDSCGNLLGR NSFEVRVCAC PGRDRRTEEE NLRKKGQSCP EPPPRSTKRA 300 |
| | | | LPTNTSSSPQ PKKKPLDGEY FTLQIRGFKR YEMFRELNDA LELKDALDGR EPGESRAHSS 360 |
| | | | HLKSKKRPSP SCHKKPMLKR EGPDSD 386 |
| 9 | TERT | Sus | MPRAPRCRAV RSLLRDRYRQ VLPLATFVRR LGPEGRRLVR RGDPAAYRAL VAQCLVCVPW 60 |
| | | scrofa | DAQPPPASPS FRQVSCLKEL VARVVQRLCE RGARNVLAFG FALLDGARGG PPVAFTTSVR 120 |
| | | | SYLPNTVTDT LRGSGAWGLL LRRVGDDVLT HLLARCALYL LVPPSCAYQV CGPPLYDLYT 180 |
| | | | AAEARPMRHK GQTPTGLGLT RPVCNGEAGR PQEQRAQGVR RRRGRAGGHP LPAKRPRHVP 240 |
| | | | EPEQGPEGQA SRAHQGRAPG PSDSDPPVMT PTRAAAKAKS REGEAPGTRH LSPQAGGARG 300 |
| | | | TCPPSWWQPH LQGKPSPHVC AAETKRFLYC SGSKEGLRRS FLLCSLPPSL AGAGRLVEVI 360 |
| | | | FLASKPGQPG ARRVPARYWR MRPLFRELLK NHARCPYKAL LRAHCPLRAA ATLSGSGGQV 420 |
| | | | CDHKVGPLAP ERLAAAAEGD SASRRLVQLL RQHSSPWQVY RLLRACLHRL VPPGLWGSPH 480 |
| | | | NKRRFLKNVK KLVSLGKHAR LSLQELMWKM KVQDCIWLRR SPDARHVQAA EHRLREAILA 540 |
| | | | KFLRWLMGTY VVELLRSFFY VTETTFQKNR LFFFRKRIWS RLQSAGIRQH LDRVRLRELS 600 |
| | | | EAEIRRRREA RPAVLTSKLR FVPKPDGLRP IVNMANVVRA RTGPGDKKVR RLTGQVKTLF 660 |
| | | | AVLNYERARR PRLLGASVLG VGDIHRAWRA FVLPLRAQDP APPLYFVKVD VTGAYDALPQ 720 |
| | | | DRLLEVVANV IRPHESTYCV RQCAVLRRTA RGHVRKSFQT HVSTFADLQP YMRQFVAHLQ 780 |
| | | | ATGPLRDAVV IEQSCSLNEA GSRLLELFLS LLRNHVIRIG GRSYVQCQGI PQGSILSTLL 840 |
| | | | CSLCYGDMEN RLFPGIQRDG VLLRLVDDFL LVTPHLTRAK AFLRTLVRGV PEYGCLANLR 900 |
| | | | KTAVNFPVED GARGGPAPLQ LPAHCLFPWC GLLLDTRTLE VHCDYASYAR TSIRASLTEN 960 |
| | | | QGFKPGRNMR RKLLAVLRLK CHGILLDLQV NSLPTVLANV YKIFLLQAYR FHACVLQLPF 1020 |
| | | | RQPLARNPSF FLRLVSDTAS CCYSLLKARN AGMSLGARGA SGPFPSEAAE WLCLHAFLLK 1080 |
| | | | LVRHRVTYSC LLGPLRAARE RLCQRLPGAT LAALEAAADP ALTTDERTIL D 1131 |
| 10 | TERT | Thunnus | MSTADTSRVL DILRSLYRHI QTLEEFADSI VFREGQKAIL IQQSDTNRFK SFVRGVFVCC 60 |
| | | albacares | DKELQQVPSC NQICTLPELL AFVLNSLKRK RKRNVLAHGY NLLSLAQEER DADHFKFQGD 120 |
| | | | VTQSAAYIHG SDLWKKVTIR LGTDITRYLF ESCSVFVTVP PSCVFQVCGI PVYDRVSMTA 180 |
| | | | ASSGFYLQSR YRTHNNTQLG RYRGVVTLKR KRNAGNPTIS KKRKRRDDRV QRGKRKRETD 240 |
| | | | QKNEEEVMLC SRKRRRVGQH EPTQETQQLC YEPAEEGQPV LVERTESLKP LENSSKGSKH 300 |
| | | | PIETTTVPLE GGPSWRSGIF PPLPPSQCFI RTLGLLYGGR GMRGFLLNRK KKSAEGSRRL 360 |
| | | | QGQDLVRIVF FEGLAYLNGL ERKPKKLPQR FFNMVPLFSQ LLRQHRRCPY SKILQRMCPL 420 |
| | | | VEERDGGQVE ISSLLPQHCG PHRVYLFVRE CLSSVIPLEL WGSVHNRLNF FARVRGFLRS 480 |
| | | | SKFERLSLAE LMWKMKVNDC DWLKISKTGR FPPSELAYRT RVLGQLLAWL LDGYVVGLVR 540 |
| | | | ACFYVTESVG QKNALKFYRQ EVWAKLLELA FRGHLSKGQM EELTPAQVAS LPKTTVISRL 600 |
| | | | RFIPKTDGMR PITRVVGADA KTRLYGGHVR DLLDMLRACV RSTPSLLGST VWGMTDIHKV 660 |
| | | | LSSLAPTQKD KPQPLYFVKV DVSGAYESLP HEKLIEVIGH ALSPVQDELF TIRRYAKIWA 720 |
| | | | DSHEGLRKSF VRQADFLEDN MGSTNMKGFV MSLQKRGVHH AILVEQYFCS DLHGREALQF 780 |
| | | | FTQMLTGSVV QFGKKTYRQC RGIPQGSVVS SLLCCLCYGH MENVLFKDIT ENKGCLMRLV 840 |
| | | | DDFLLITPDL HEAQSFLKIL LAGVPQYGLV VNPQKVVINF QASGGMDSCP GIRILPPHCL 900 |
| | | | FPWCGLLLDT HSLDVYKDYS SYAGLSLRYS LTLGSFHSPG QQMRRKLMAI LRLKCHALFL 960 |
| | | | DLKTNSLEAV YKNIYKLVLL HAYRFHVCAQ SLPFGQTVAK NPVYFLQMIW DMAEYANELI 1020 |
| | | | RLSNKGLMLG SKAQTGIVQY EAVELLFCLS FLLVLSQHRS HYKDLLLHLH KRKRSLERCL 1080 |
| | | | GDLRLARVRQ ATNPRTPVDF LAIQT 1105 |
| 11 | TERT | Syn- | EARPALLTSR LRFIPK 16 |
| | extension | thetic | |
| | inducing | | |
| | peptide | | |
| 12 | CDK4 | Thunnus | MAHGSSIQYE PVAEIGGGAY GTVYKARDTE SGQFVALKSV RVQTDQNGLP VSTVREVALL 60 |
| | | albacares | RRLEQFDHPN VVRLMDVCAT QRSDQETKVT LVFEHVDQDL KTYLERAPAP GLSPDRIKDL 120 |
| | | | MRQLLCGLAF LHSNRVMHRD LKPENILVTS QGQVKLADFG LARIYSCHMA LTPVVVTLWY 180 |
| | | | RPPEVLLQSS YATPVDIWST GCIFAEMERR KPLFCGDSEV DQLGKIFQVF GLPPEEEWPT 240 |
| | | | DVTLSRKNFP PLFPRPITDF VPEINEQGAQ LLLKMLTFDP FKRISALNAL EHPYFRDEET 300 |
| | | | LTQTKS 306 |
| 13 | p15 siRNA 1 | Syn- | gggauauuua ggagugugut t 21 |
| | | thetic | |
| 14 | p15 siRNA 2 | Syn- | acacacuccu aaauauccct g 21 |
| | | thetic | |
| 15 | p15 siRNA 3 | Syn- | ggucagacua agaaauauut t 21 |
| | | thetic | |
| 16 | p15 siRNA 4 | Syn- | aauauuucuu agucugacct c 21 |
| | | thetic | |
| 17 | p16 siRNA 1 | Syn- | cgcaccgaau aguuacggut t 21 |
| | | thetic | |
| 18 | p16 siRNA 2 | Syn- | accguaacua uucggugcgt t 21 |
| | | thetic | |
| 19 | p18 siRNA | Syn- | agaccgaacu gguuucgcu 19 |
| | | thetic | |
| 20 | p53 siRNA | Syn- | gaagaaaatt tccgcaaaa 19 |
| | | thetic | |
| 21 | Adenovirus | Syn- | MRHIICHGGV ITEEMAASLL DQLIEEVLAD NLPPPSHFEP PTLHELYDLD VTAPEDPNEE 60 |
| | serotype | thetic | AVSQIFPDSV MLAVQEGIDL LTFPPAPGSP EPPHLSRQPE QPEQRALGPV SMPNLVPEVI 120 |
| | 2 E1 | | DLTCHEAGFP PSDDEDEEGE EFVLDYVEHP GHGCRSCHYH RRNTGDPDIM CSLCYMRTCG 180 |
| | | | MFVYSPVSEP EPEPEPEPEP ARPTRRPKLV PAILRRPTSP VSRECNSSTD SCDSGPSNTP 240 |
| | | | PEIHPVVPLC PIKPVAVRVG GRRQAVECIE DLLNESGQPL DLSCKRPRP 289 |
| 22 | Adenovirus | Syn- | MRHIICHGGV ITEEMAASLL DQLIEEVLAD NLPPPSHFEP PTLHELYDLD VTAPEDPNEE 60 |
| | serotype | thetic | AVSQIFPDSV MLAVQEGIDL LTFPPAPGSP EPPHLSRQPE QPEQRALGPV SMPNLVPEVI 120 |
| | 5 E1 | | DLTCHEAGFP PSDDEDEEGE EFVLDYVEHP GHGCRSCHYH RRNTGDPDIM CSLCYMRTCG 180 |
| | | | MFVYSPVSEP EPEPEPEPEP ARPTRRPKMA PAILRRPTSP VSRECNSSTD SCDSGPSNTP 240 |
| | | | PEIHPVVPLC PIKPVAVRVG GRRQAVECIE DLLNEPGQPL DLSCKRPRP 289 |
| 23 | Human | Syn- | MHQKRTAMFQ DPQERPRKLP QLCTELQTTI HDIILECVYC KQQLLRREVY DFAFRDLCIV 60 |
| | papilloma- | thetic | YRDGNPYAVC DKCLKFYSKI SEYRHYCYSL YGTTLEQQYN KPLCDLLIRC INCQKPLCPE 120 |
| | virus | | EKQRHLDKKQ RFHNIRGRWT GRCMSCCRSS RTRRETQL 158 |
| | (HPV) | | |
| | E6 type | | |
| | 16 | | |
| 24 | Human | Syn- | MARFEDPTRR PYKLPDLCTE LNTSLQDIEI TCVYCKTVLE LTEVFEFAFK DLFVVYRDSI 60 |
| | papilloma- | thetic | PHAACHKCID FYSRIRELRH YSDSVYGDTL EKLTNTGLYN LLIRCLRCQK PLNPAEKLRH 120 |
| | virus | | LNEKRRFHNI AGHYRGQCHS CCNRARQERL QRRRETQV 158 |
| | (HPV) | | |
| | E6 type | | |
| | 18 | | |
| 25 | Human | Syn- | MHGDTPTLHE YMLDLQPETT DLYCYEQLND SSEEEDEIDG PAGQAEPDRA HYNIVTFCCK 60 |
| | papilloma- | thetic | CDSTLRLCVQ STHVDIRTLE DLLMGTLGIV CPICSQKP 98 |
| | virus | | |
| | (HPV) E7 | | |
| | 16 | | |
| 26 | Human | Syn- | MHGPKATLQD IVLHLEPQNE IPVDLLCHEQ LSDSEEENDE IDGVNHQHLP ARRAEPQRHT 60 |
| | papilloma- | thetic | |
| | virus | | MLCMCCKCEA RIKLVVESSA DDLRAFQQLF LNTLSFVCPW CASQQ 105 |
| | (HPV) E7 | | |
| | type 18 | | |
| 27 | c-myc | | MPLNVSFANK NYDLDYDSVQ PYFYCDEEEN FYHQQQQSEL QPPAPSEDIW KKFELLPTPP 60 |
| | | Bos | LSPSRRSGLC SPSYVAVASF SPRGDDDGGG GSFSSADQLE MVTELLGGDM VNQSFICDPD 120 |
| | | taurus | DETLIKNIII QDCMWSGFSA AAKLVSEKLA SYQAARKDGG SPSPARGHGG CSTSSLYLQD 180 |
| | | | LSAAASECID PSVVFPYPLN DSSSPKPCAS PDSTAFSPSS DSLLSSAESS PRASPEPLAL 240 |
| | | | HEETPPTTSS DSEEEQEDEE EIDVVSVEKR QPPAKRSESG SPSAGSHSKP PHSPLVLKRC 300 |
| | | | HVSTHQHNYA APPSTRKDYP AAKRAKLDSG RVLKQISNNR KCASPRSSDT EENDKRRTHN 360 |
| | | | VLERQRRNEL KRSFFALRDQ IPELENNEKA PKVVILKKAT AYILSVQAEQ QKLKSEIDVL 420 |
| | | | QKRREQLKLK LEQIRNSCA 439 |
| 28 | Feline | Syn- | MSGASSGTAI GAKLFGISSV LGEYRVLIGD EGAGPSRSPS EVSFSVWYQS RAARLVTLCL 60 |
| | leukemia | thetic | VASFLVPCLT FLIAEAVMGP GSHRRERPSS GVFHLGAPLN VSFANRNYDL DYDSVQPYFY 120 |
| | virus | | CDEEENFYQQ QQQSELQPPA PSEDIWKKFE LLPTPPLSPS RRSGLCSPSY VAFASFSPRG 180 |
| | v-myc | | DDDGGGGSFS TADQLEMVTE LLGGDMVNQS FICDPDDETF IKNIIIQDCM WSGFSAAAKL 240 |
| | | | VSEKLASYQA ARKDSGSPSP ARGPGGCPTS SLYLQDLTAA ASECIDPSVV FPYPLNDSSS 300 |
| | | | PKPCASPDSA AFSPSSDSLL SSAESSPRAS PEPLALHEET PPTTSSDSEE EQEEEKEIDV 360 |
| | | | VSVEKRQPPA KRSESGSPSA GGHSKPPHSP LVLKRCHVPT HQHNYAAPPS TRKDYPAAKR 420 |
| | | | AKLDSGRVLK QISNNRKCIS PRSSDTEEND KRRTHNVLER QRRNELKRSF FALRDQIPEL 480 |
| | | | ENNEKAPKVV ILKKATAYIL SVQAGEQKLI SEKDLLRKRR EQLKHKLEQL RNSCA 535 |
| 29 | Polycomb | Bos | MHRTTRIKIT ELNPHLMCVL CGGYFIDATT IIECLHSFCK TCIVRYLETS KYCPICDVQV 60 |
| | complex | taurus | HKTRPLLNIR SDKTLQDIVY KLVPGLFKNE MKRRRDFYAA HPSADAANGS NEDRGEVADE 120 |
| | protein | | DKRIITDDEI ISLSIEFFDQ NRLDRKINKD KEKSKEEVND KRYLRCPAAM TVMHLRKFLR 180 |
| | BMI-1 | | SKMDIPNTFQ IDVMYEEEPL KDYYTLMDIA YIYTWRRNGP LPLKYRVRPT CKRMKISHQR 240 |
| | | | DGLTNTGELE SDSGSDKANS PAGGIPSTSS CLPSPSTPVQ SPHPQFPHIS STMNGTSSSP 300 |
| | | | SGNHQSSFAN RPRKSSVNGS SATSSG 326 |
| 30 | Puromycin | Syn- | MTEYKPTVRL ATRDDVPRAV RTLAAAFADY PATRHTVDPD RHIERVTELQ ELFLTRVGLD 60 |
| | resistance | thetic | IGKVWVADDG AAVAVWTTPE SVEAGAVFAE IGPRMAELSG SRLAAQQQME GLLAPHRPKE 120 |
| | | | PAWFLATVGV SPDHQGKGLG SAVVLPGVEA AERAGVPAFL ETSAPRNLPF YERLGFTVTA 180 |
| | | | DVEVPEGPRT WCMTRKPGA 199 |
| 31 | Green | | MSKGEELFTG VVPILVELDG DVNGHKFSVR GEGEGDATNG KLTLKFICTT GKLPVPWPTL 60 |
| | Fluorescent | | VTTLTYGVQC FSRYPDHMKR HDFFKSAMPE GYVQERTISF KDDGTYKTRA EVKFEGDTLV 120 |
| | protein | | NRIELKGIDF KEDGNILGHK LEYNFNSHNV YITADKQKNG IKANFKIRHN VEDGSVQLAD 180 |
| | (GFP) | | HYQQNTPIGD GPVLLPDNHY LSTQSVLSKD PNEKRDHMVL LEFVTAAGIT HGMDELYK 238 |
| 32 | Kras | Bos | MTEYKLVVVG AGGVGKSALT IQLIQNHFVD EYDPTIEDSY RKQVVIDGET CLLDILDTAG |
| | (bovine0 | taurus | QEEYSAMRDQ YMRTGEGFLC VFAINNTKSF EDIHHYREQI KRVKDSEDVP MVLVGNKCDL |
| | | | PSRTVDTKQA QDLARSYGIP FIETSAKTRQ |
| | | | 160 170 180 |
| | | | RVEDAFYTLV REIRQYRLKK ISKEEKTPGC VKIKKCIVM |

1. A method of expanding a population of cells in culture comprising:
(a) delivering one or more transient immortalizing factors to the population of cells in an amount sufficient to temporarily induce immortalization in the cell;
(b) culturing the cells of step (a) with the one or more immortalizing factor for a sufficient time to allow for cell proliferation.

2. The method of claim 1, wherein the population of cells produced in step (b) do not have a modified genome.

3. The method of claim 1, wherein the one or more transient immortalizing factors comprise telomerase reverse transcriptase (TERT), cyclin-dependent kinase 4 (CDK4), SV40 T antigen, Epstein-Barr virus (EBV), adenovirus E1 protein, human papillomavirus (HPV) E6 protein, HPV E7 protein, c-myc, v-myc, Ras or a small molecule.

4. The method of claim 1, wherein the one or more transient immortalizing factors comprise an inhibitor of p15, p16, p27, p18, or p53.

5. (canceled)

6. The method of claim 1, wherein the one or more transient immortalizing factors further comprises CRISPRa or CRISPRi.

7. The method of claim 1, wherein the population of cells comprises primary cells.

8. The method of claim 7, wherein the population of cells comprises muscle cells.

9. The method of claim 8, wherein the muscle cells comprise muscle satellite cells.

10. The method of claim 9, wherein the cells comprise bovine cells.

11. The method of claim 1, wherein further comprising (c) culturing the population of cells produced after step (b) in culture conditions without the one or more immortalizing factor for a sufficient time to produce cells without the exogenous immortalizing factor.

12. (canceled)

13. The method of claim 12, wherein the one or more transient immortalizing factors is selected from the group consisting of (a) telomerase reverse transcriptase (TERT) and targets cyclin-dependent kinase 4 (CDK4); (b) TERT and BmiI (p16 inhibitor); (c) TERT+cell cycle inhibitor (p15, p16, CDK4, BMi1, etc.); (d) telomerase extending factor and factor that overcomes G1-S phase cell cycle checkpoint (e.g., p15 inhibitor, p16 inhibitor, CDK3, Bmi1, etc) and (e) combinations thereof.

14-15. (canceled)

16. The method of claim 1, wherein the method produces an in vitro derived cell population.

17. A foodstuff comprising the in vitro derived cell population of claim 16, wherein the cells are not genetically modified.

18. A method for large scale production of in vitro cultured meat product comprising a population of muscle cells, the method comprising:
(a) delivering one or more transient immortalizing factors to the population of cells in an amount sufficient to temporarily induce immortalization in the cell;
(b) culturing the cells of step (a) with the one or more immortalizing factor for a sufficient time to allow for cell proliferation; and
(c) culturing the population of cells produced after step (b) in culture conditions without the one or more immortalizing factor for a sufficient time to produce cells without the exogenous immortalizing factor.

19. The method of claim 18, wherein the one or more transient immortalizing factors comprise telomerase reverse transcriptase (TERT), cyclin-dependent kinase 4 (CDK4), Bmi1, or SV40 T antigen.

20. The method of claim 18, wherein the one or more transient immortalizing factors comprises an inhibitor of a factor of cellular senescence comprising inhibitors of p15, p16, p27, p18, or p53.

21. (canceled)

22. The method of claim 18, wherein the one or more transient immortalizing factors further comprises CRISPRa or CRISPRi.

23. The method of claim 18, wherein the population of cells comprises primary cells.

24. The method of claim 23, wherein the population of cells comprises muscle cells.

25. The method of claim 18, wherein the muscle cells comprise muscle satellite cells.

26-27. (canceled)
